(19) United States
(12) Patent Application Publication
(10) Pub. No.: US 2020/0038463 A1
(43) Pub. Date: Feb. 6, 2020

(54) METHODS AND COMPOSITIONS OF MMA CONSTRUCTS AND VECTORS

(71) Applicant: Selecta Biosciences, Inc., Watertown, MA (US)

(72) Inventors: Peter Keller, Beverly, MA (US); Takashi Kei Kishimoto, Lexington, MA (US)

(73) Assignee: Selecta Biosciences, Inc., Watertown, MA (US)

(21) Appl. No.: 16/513,576

(22) Filed: Jul. 16, 2019

Related U.S. Application Data
(60) Provisional application No. 62/839,761, filed on Apr. 28, 2019.
(60) Provisional application No. 62/698,528, filed on Jul. 16, 2018.

(51) Int. Cl. A61K 35/761; C12N 7/00; C12N 9/90; A61K 47/69; A61P 3/00; A61K 31/436; A61P 37/06

(52) U.S. Cl. CPC A61K 35/761; C12N 7/00; C12N 9/90; C12Y 504/99002; C12N 2750/14143; A61P 3/00; A61K 31/436; A61P 37/06; A61K 47/6931

(57) ABSTRACT

Provided herein are methods and compositions related to nucleic acids encoding methylmalonyl-CoA mutase (MUT) as well as related vectors, such as AAV vectors and Anc80 vectors. Also, provided are methods for administering viral vectors that comprise a sequence that encodes an enzyme associated with an organic acidemia and an expression control sequence, in combination with synthetic nanocarriers coupled to an immunosuppressant.

### RELATED APPLICATIONS

This application claims the benefit under 35 U.S.C. § 119(e) of U.S. Provisional Application Ser. No. 62/698,528, filed on Jul. 16, 2018 and U.S. Provisional Application Ser. No. 62/839,761, filed Apr. 28, 2019, the entire contents of each of which are incorporated herein by reference.

### FIELD OF THE INVENTION

The invention relates to methods and compositions related to nucleic acids encoding methylmalonyl-CoA mutase (MUT) as well as related vectors, such as AAV, Anc80 and AAV/Anc80 vectors. Also, provided are methods for administering viral vectors that comprise a sequence that encodes an enzyme associated with an organic acidemia, such as methylmalonic acidemia, and an expression control sequence, in combination with synthetic nanocarriers coupled to an immunosuppressant.

### SUMMARY OF THE INVENTION

Provided herein are methods and compositions related to nucleic acids encoding methylmalonyl-CoA mutase (MUT) as well as related viral vectors. Also, provided herein are methods and compositions for administering the viral vectors that comprise a sequence that encodes an enzyme associated with an organic academia, such as methylmalonic acidemia (MMA), and one or more expression control sequences, in combination with synthetic nanocarriers coupled to an immunosuppressant. In an embodiment of any one of the compositions or methods provided herein, the viral vector encodes a wild-type MUT. The administration of the viral vector in combination with synthetic nanocarriers coupled to an immunosuppressant may have a therapeutic benefit for any one of the purposes provided herein in any one of the methods or compositions provided herein.

In another aspect a method or composition as described in any one of the Examples is provided. In one embodiment, the compositions comprise any one of the viral vectors provided in the Examples.

In another aspect, any one of the compositions is for use in any one of the methods provided.

In another aspect, any one of the methods or compositions is for use in treating any one of the diseases or disorders described herein. In another aspect, any one of the methods or compositions is for use in reducing an immune response (i.e., humoral and/or cellular) to a viral antigen and/or the expressed product of a viral vector, increasing expression of the sequence encoding the enzyme, or for repeated administration of a viral vector.

### BRIEF DESCRIPTION OF THE FIGURES

FIG. 1 is a schematic depicting an exemplary mRNA construct.

FIG. 2 depicts a liver-specific construct, comprising apoE-hAAT-synMUT4. The abbreviations are as follows: apolipoprotein E, ApoE; hAAT, human alpha-1-antitrypsin; HBB-2, hemoglobin subunit beta-2.

FIG. 3 is a schematic depicting exemplary constitutive promoter constructs comprising EFα long-synMUT4. The bottom schematic shows an instance where the promoter (EF1α, elongation factor 1 alpha 1) and the transgene (synMUT4) are separated by an intron (HBB-2, hemoglobin subunit beta-2). The top schematic illustrates the same construct without the intron. Both constructs were formed in rAAV8 and in Anc80.

FIG. 4 is a schematic depicting exemplary liver-specific promoter constructs comprising apoE-hAAT (short)-HBB2-synMUT4. Each either contains or does not contain an intron (HBB-2, hemoglobin subunit beta-2) and/or a transcriptional regulatory element (human post-transcriptional regulatory element, HPRE).

FIG. 5 is a schematic depicting exemplary constitutive promoter constructs comprising apoE-hAAT (short)-HBB2-synMUT4. Each either contains or does not contain an intron (HBB-2, hemoglobin subunit beta-2) and/or a transcriptional regulatory element (human post-transcriptional regulatory element, HPRE).

FIG. 6 is a schematic depicting two constitutive primer constructs comprising EF1α (short)-syn MUT4. Both have synthetic (SYN) introns between the promoter and transgene, and the lower schematic shows a regulatory element (HPRE) between the transgene and the poly A tail.

FIG. 7 shows FACS results of plasmid DNA in Huh7 cells, 48 hours after transfection, using different constructs: Huh7 cell control, rAAV2-CB7-CI-eGFP-WPRE-rBG MOI 1E4, AAV2/2-CMV-EGFP-WPRE.bGH (A646) MOI 1E4, AAV2/2-CMV-EGFP-WPRE.bGH (A646) MOI 1E5, AAV2/Anc80 AAP.-CMV-EGFP-WPRE.bGH (A915) MOI 1E5, AAV2/Anc80 AAP.-CMV-EGFP-WPRE.bGH (A915) MOI 1E6, and AAV2/Anc80 AAP.-CMV-EGFP-WPRE.bGH (A915) MOI 2E6.

FIG. 8 is a graph depicting synMUT4 expression in liver samples.

FIG. 9 is a graph depicting synMUT4 expression in kidney samples.

FIG. 10 is a graph showing biodistribution of synMUT4 in liver.

FIG. 11 is a schematic illustrating the process of analytical ultracentrifugation to determine the Anc80 reference standard.

FIG. 12 shows the graphical results of the Anc80 reference standard determination described in FIG. 11.

FIG. 13 shows the levels of methylmalonic acid (MMA) and alkaline phosphatase (ALK) in methylmalonyl-CoA mutase (MUT) deficient mice after administration of 300 μg ImmTOR nanoparticles. (D0=day 0, administration; D12=day 12, D30=day 30; ns=not significant; *=P<0.05).

FIG. 14 shows the weight gain in methylmalonyl-CoA mutase (MUT) deficient mice after administration of an Anc80-MUT vector (2.5×10¹² vg/kg) and 100 μg or 300 μg immune tolerance-inducing synthetic nanoparticles (ImmTOR).

FIG. 15 shows the levels of anti-Anc80 antibodies in MUT deficient mice after administration of the Anc80-luciferase (Anc80-Luc) vector (5.0×10¹⁰ vg/kg) or co-administration of the Anc80 AAV vector and 100 μg ImmTOR nanoparticles. (D0=day 0, administration or co-administration; D14=day 14; D28=day 28; n=5/group).

FIG. 16 shows the levels of methylmalonic acid (MMA) in MUT deficient mice after 14 and 30 days after administration of an Anc80-MUT vector (2.5×10¹² vg/kg) or co-administration of the Anc80-MUT vector and 100 μg or 300 μg ImmTOR nanoparticles. (n=11-14 mice/group for 14 day group; n=12-14 mice/group for 30 day group; *=P<0.05; **=P<0.01; ***=P<0.005).

FIG. 17 shows the number of Anc80-MUT vector genomes per cell in MUT deficient mice after administration of the Anc80-MUT vector (2.5×10¹² vg/kg) or co-administration of the Anc80-MUT vector and 100 μg or 300 μg ImmTOR nanoparticles. (n=4-6 mice; M=male mouse, F=female mouse; *=P<0.05).

FIG. 18 shows the weight gain in MUT deficient mice after a second administration of the Anc80-MUT vector (2.5×10¹² vg/kg) or co-administration of the Anc80-MUT vector and 100 μg ImmTOR nanoparticles. (Anc80 only n=7 mice; Anc80+ImmTOR n=9 mice; *=P<0.05; **=P<0.001).

FIG. 19 shows the levels of anti-Anc80 antibodies (IgG) in MUT deficient mice after a 1^{st} and a 2^{nd} dose of the Anc80-MUT vector or co-administration of the Anc80-MUT vector and 100 μg or 300 μg ImmTOR nanoparticles.

FIG. 20 shows the levels of methylmalonic acid (MMA) in MUT deficient mice after a 1^{st} and a 2^{nd} dose of the Anc80-MUT vector or co-administration of the Anc80-MUT vector and 100 μg or 300 μg ImmTOR nanoparticles (Anc80 only n=13 mice; Anc80+100 μg ImmTOR n=15; Anc80+300 μg ImmTOR n=12).

FIG. 21 shows the percentage of methylmalonic acid (MMA) in MUT deficient mice after a 1^{st} and a 2^{nd} dose of the Anc80-MUT vector or co-administration of the Anc80-MUT vector and 100 μg or 300 μg ImmTOR nanoparticles. The 2^{nd} dose was administered on day 56. (n=4-7, *=P<0.05; **=P<0.01).

FIG. 22 shows one dosing scheme for co-administration of a first dose of the Anc80-Luc AAV vector (5.0×10¹⁰ vg/kg) and 300 μg ImmTOR nanoparticles followed by a second dose of the Anc80 AAV vector (2.5×10¹² vg/kg) and a second dosing scheme for co-administration of a first dose of the Anc80-Luc AAV vector (5.0×10¹⁰ vg/kg) and 300 μg ImmTOR nanoparticles followed by a second dose of the Anc80 AAV vector (2.5×10¹² vg/kg) and 300 μg ImmTOR nanoparticles. The first administration is at day 0 (d0) and the second administration is at day 47 (d47). (MMA=methylmalonic acid; Abs=absorbance; n=6/group).

FIG. 23 shows the levels of anti-Anc80 antibodies in MUT deficient mice after administration of the first or the second dosing scheme of FIG. 22. (d0=day 0, day of first dose; d12=day 12; d30=day 30; 2-d1 (day of second dose; 47 days after first dose); 2-d12 (12 days after second dose); 2-d30 (30 days after second dose).

FIG. 24 shows the levels of methylmalonic acid (MMA) in MUT deficient mice after administration of the second dose as described in FIG. 22. The Luc-SVP+wtMUT group received a second dose comprising the Anc80 AAV vector (2.5×10¹² vg/kg) alone, whereas the Luc-SVP+wtMUT-ImmTOR 300 μg group received a second dose comprising the Anc80 AAV vector (2.5×10¹² vg/kg) and 300 μg ImmTOR nanoparticles (2-D0=day of second dose, 47 days after first dose; 2-D12=12 days after second dose; ****=P<0.001).

FIG. 25 shows the levels of methylmalonic acid (MMA) in MUT deficient mice after administration of the first or the second dosing scheme of FIG. 22. (D0=day 0, day of the first dose; D12=12 days after the first dose; D30=30 days after the first dose; 2-D0=day of second dose, 47 days after first dose; 2-D12=12 days after second dose; *=P<0.05; **=P<0.01; ****=I′<0.001).

FIG. 26 shows the levels of anti-Anc80 antibodies (IgG) in MUT deficient mice with maternally transferred anti-Anc80 antibodies prior to after administration of the Anc80-MUT vector (n=17).

FIG. 27 shows the levels of methylmalonic acid (MMA) in MUT deficient mice with maternally transferred anti-Anc80 antibodies after administration of the Anc80 vector (5.0×10¹² vg/kg) or co-administration of the Anc80-MUT vector and 100 μg or 300 μg ImmTOR nanoparticles. (“X” indicates that a mouse died, n=3 mice for Anc80-MUT only, 5 for Anc80-MUT+100 μg ImmTOR, 7 for Anc80-MUT+300 μg ImmTOR).

FIG. 28 shows the levels of methylmalonic acid (MMA) in MUT deficient mice with maternally transferred anti-Anc80 antibodies after a 1^{st} and a 2^{nd} dose of the Anc80-MUT vector or co-administration of the Anc80-MUT vector and 100 μg or 300 μg ImmTOR nanoparticles. (“X” indicates that a mouse died, n=3 mice for Anc80-MUT only, 5 for Anc80-MUT+100 μg ImmTOR, 7 for Anc80-MUT+300 μg ImmTOR).

FIG. 29 shows the levels of FGF21 in MUT deficient mice with maternally transferred anti-Anc80 antibodies after a 1st and a 2nd dose of the Anc80-MUT vector or co-administration of the Anc80-MUT vector and 100 μg or 300 μg ImmTOR nanoparticles (“X” indicates that a mouse died, n=6 mice for Anc80-MUT only, 7 for Anc80-MUT+100 μg ImmTOR, 10 for Anc80-MUT+300 μg ImmTOR) with the numbers of mice in each group surviving to the 2nd injection on day 21 and to day 30 shown in parentheses.

FIG. 30 shows the levels of anti-Anc80 antibodies (IgG) in MUT deficient mice with maternally transferred anti-Anc80 antibodies after a 1^{st} and a 2^{nd} dose of the Anc80-MUT vector or co-administration of the Anc80-MUT vector and 100 μg or 300 μg ImmTOR nanoparticles.

FIG. 31 shows the weight gain in MUT deficient mice after a 1^{st} and a 2^{nd} administration of the Anc80-MUT vector (5.0×10¹² vg/kg) or co-administration of the Anc80-MUT vector and 100 μg ImmTOR nanoparticles. (“X” indicates that a mouse died, n=3 mice for Anc80-MUT only, 5 for Anc80-MUT+100 μg ImmTOR, 7 for Anc80-MUT+300 μg ImmTOR).

| Abbreviation | Signification |
|---|---|
| ITR-2 | Inverted Terminal Repeat 2 |
| ApoE | Apolipoprotein E |
| hAAT long | Human Alpha-1-antitrypsin |
| HBB-2 | Hemoglobin subunit beta-2 |
| synMUT4 | Synthetic Methyl malonyl CoA Mutase 4 |
| Poly A | Polyadenylation |
| Abx: Kana | Kanamycin antibody |
| EF1α long | Elongation factor 1 alpha 1 |
| HPRE | Human Post Transcriptional Regulatory |
| SYN | Synthetic intron |
| Huh-7 | Human liver cell |
| CB-7 | Chicken β actin |
| EGFP | Engineered green fluorescent protein |
| WPRE | Woodchuck Hepatitis Virus Post Transcriptional Regulatory |
| | Element |
| CMV | Cytomegalovirus |
| MOI | Multiplicity Og Infection |

### DETAILED DESCRIPTION OF THE INVENTION

Before describing the present invention in detail, it is to be understood that this invention is not limited to particularly exemplified materials or process parameters as such may, of course, vary. It is also to be understood that the terminology used herein is for the purpose of describing particular embodiments of the invention only, and is not intended to be limiting of the use of alternative terminology to describe the present invention.

All publications, patents and patent applications cited herein, whether supra or infra, are hereby incorporated by reference in their entirety for all purposes. Such incorporation by reference is not intended to be an admission that any of the incorporated publications, patents and patent applications cited herein constitute prior art.

As used in this specification and the appended claims, the singular forms “a,” “an” and “the” include plural referents unless the content clearly dictates otherwise. For example, reference to “a polymer” includes a mixture of two or more such molecules or a mixture of differing molecular weights of a single polymer species, reference to “a synthetic nanocarrier” includes a mixture of two or more such synthetic nanocarriers or a plurality of such synthetic nanocarriers, reference to “a DNA molecule” includes a mixture of two or more such DNA molecules or a plurality of such DNA molecules, reference to “an immunosuppressant” includes a mixture of two or more such immunosuppressant molecules or a plurality of such immunosuppressant molecules, and the like.

As used herein, the term “comprise” or variations thereof such as “comprises” or “comprising” are to be read to indicate the inclusion of any recited integer (e.g. a feature, element, characteristic, property, method/process step or limitation) or group of integers (e.g. features, elements, characteristics, properties, method/process steps or limitations) but not the exclusion of any other integer or group of integers. Thus, as used herein, the term “comprising” is inclusive and does not exclude additional, unrecited integers or method/process steps.

In embodiments of any of the compositions and methods provided herein, “comprising” may be replaced with “consisting essentially of” or “consisting of”. The phrase “consisting essentially of” is used herein to require the specified integer(s) or steps as well as those which do not materially affect the character or function of the claimed invention. As used herein, the term “consisting” is used to indicate the presence of the recited integer (e.g. a feature, element, characteristic, property, method/process step or limitation) or group of integers (e.g. features, elements, characteristics, properties, method/process steps or limitations) alone.

### A. INTRODUCTION

Organic acidemia (organic aciduria) describes a group of metabolic disorders in which normal amino acid metabolism is disrupted. The disorders generally result in the accumulation of amino acids which are not normally present, and are typically caused by disruptions of the metabolism of branched-chain amino acids, such as isoleucine, leucine, and valine. There are four main types of organic acidemia: methylmalonic acidemia, propionic acidemia, isovaleric acidemia, and maple syrup urine disease. Methylmalonic acidemia (MMA) is a common and severe organic acidemia frequently caused by mutations in methylmalonyl-CoA mutase (MUT). MMA is an autosomal recessive disorder and results in a build-up of methylmalonic acid. Severely affected patients can benefit from liver transplantation and may require kidney transplantation due to renal failure.

As examples, a series of Anc80 and AAV vector constructs expressing human MUT4 transgene were developed. The Anc80 vectors that used either a liver-specific promoter or constitutive promoter in wild-type mice were found to have similar levels of MUT expression, while Anc80 vectors with constitutive promoters also showed significant expression in the kidney. After treatment with Anc80 or AAV8 vectors at doses between 5×10¹¹ and 6×10¹² GC/kg, a hypomorphic murine model of MMA displayed improved growth, reduced levels of circulating metabolites, and increased MUT enzyme activity. The effects of AAV gene therapy with all vectors was apparent by 12 days, and persisted for at least one year. Furthermore, Anc80 vectors administered to neonatal mice (1×10¹³ GC/kg) were found to be effective in rescuing a murine model of MMA with a neonatal-lethal phenotype. Compositions comprising any one of such constructs are provided herein in some aspects. Any one of such constructs can be used in any one of the methods and compositions provided herein.

In addition, it is noted that while viral vectors are promising therapeutics for a variety of applications such as transgene expression, cellular and humoral immune responses against the viral vector can diminish efficacy and/or reduce the ability to use such therapeutics, particularly in a repeat administration context. In fact, cellular and humoral immune responses against a viral transfer vector can develop after a single administration of the viral transfer vector. These immune responses include antibody, B cell and T cell responses and can be specific to viral antigens of the viral vector, such as viral capsid or coat proteins or peptides thereof. After viral vector administration, neutralizing antibody titers can increase and remain high for several years and can reduce the effectiveness of readministration of the viral vector, as repeated administration of a viral transfer vector generally results in enhanced immune responses. Moreover, for many therapeutic applications, it is anticipated that multiple rounds of administration of viral vectors will be needed for long-term benefits, and, without the methods and compositions provided herein, the ability to do so would be expected to be severely limited particularly if readministration is needed.

Provided are adeno-associated virus (AAV) vectors and Anc80 vectors encoding MUT, including a wild-type MUT, the MUT4 gene, etc. for administration in combination with biodegradable synthetic nanocarriers containing an immunosuppressant, such as rapamycin. Such a combination can be made and used to prevent immune responses, such as antibody responses. Thus, provided herein are methods and compositions for treating a subject with an AAV vector, an Anc80 or an AAV/Anc80 vector comprising a sequence encoding a wild-type MUT or any one of the constructs provided herein in combination with synthetic nanocarriers comprising an immunosuppressant.

Thus, the inventors have surprisingly and unexpectedly discovered that the problems and limitations noted above can be overcome by practicing the invention disclosed herein. Methods and compositions are provided that offer solutions to the aforementioned obstacles to effective use of the viral vectors for treatment.

The invention will now be described in more detail below.

### B. DEFINITIONS

“Administering” or “administration” or “administer” means giving or dispensing a material to a subject in a manner that is pharmacologically useful. The term is intended to include “causing to be administered”. “Causing to be administered” means causing, urging, encouraging, aiding, inducing or directing, directly or indirectly, another party to administer the material. Any one of the methods provided herein may comprise or further comprise a step of administering concomitantly viral vector and synthetic nanocarriers comprising an immunosuppressant. In some embodiments, the concomitant administration is performed repeatedly. In still further embodiments, the concomitant administration is simultaneous administration. “Simultaneous” means administration at the same time or substantially at the same time where a clinician would consider any time between administrations virtually nil or negligible as to the impact on the desired therapeutic outcome. In some embodiments, “simultaneous” means that the administrations occur with 5, 4, 3, 2, 1 or fewer minutes.

“Amount effective” in the context of a composition or dosage form for administration to a subject as provided herein refers to an amount of the composition or dosage form that produces one or more desired results in the subject, for example, the reduction or elimination of an immune response against a viral vector or an expression product thereof and/or efficacious transgene expression. The amount effective can be for in vitro or in vivo purposes. For in vivo purposes, the amount can be one that a clinician would believe may have a clinical benefit for a subject. In any one of the methods provided herein, the composition(s) administered may be in any one of the amounts effective as provided herein.

Amounts effective can involve reducing the level of an undesired immune response, although in some embodiments, it involves preventing an undesired immune response altogether. Amounts effective can also involve delaying the occurrence of an undesired immune response. An amount effective can also be an amount that results in a desired therapeutic endpoint or a desired therapeutic result. Amounts effective, in some embodiments, result in a tolerogenic immune response in a subject to an antigen, such as a viral antigen of the viral vector and/or expressed product. Amounts effective also can result in increased transgene expression (the transgene being delivered by the viral vector). This can be determined by measuring transgene protein concentrations in various tissues or systems of interest in the subject. This increased expression may be measured locally or systemically. The achievement of any of the foregoing can be monitored by routine methods.

In some embodiments of any one of the compositions and methods provided, the amount effective is one in which the desired immune response, such as the reduction or elimination of an immune response, persists in the subject for at least 1 week, at least 2 weeks or at least 1 month. In other embodiments of any one of the compositions and methods provided, the amount effective is one which produces a measurable desired immune response, such as the reduction or elimination of an immune response. In some embodiments, the amount effective is one that produces a measurable desired immune response, for at least 1 week, at least 2 weeks or at least 1 month.

Amounts effective will depend, of course, on the particular subject being treated; the severity of a condition, disease or disorder; the individual patient parameters including age, physical condition, size and weight; the duration of the treatment; the nature of concurrent therapy (if any); the specific route of administration and like factors within the knowledge and expertise of the health practitioner. These factors are well known to those of ordinary skill in the art and can be addressed with no more than routine experimentation.

In general, doses of the synthetic nanocarriers coupled to an immunosuppressant described herein can range from about 1 μg/kg to about 100,000 μg/kg. In some embodiments, the doses can range from about 0.01 mg/kg to about 100 mg/kg. In still other embodiments, the doses can range from about 1 μg/kg to 100 μg/kg, from about 0.01 mg/kg to about 10 mg/kg, about 25 mg/kg to about 50 mg/kg, about 50 mg/kg to about 75 mg/kg or about 75 mg/kg to about 100 mg/kg. In general, doses of the vectors described herein can range from 1*10⁸-1*10¹⁴ VG/kg. In some embodiments, the doses can range from about 1*10⁹-1*10¹³ VG/kg. In still other embodiments, the doses can range from about 1*10⁹-1*10¹¹ VG/kg or from about 1*10¹¹-1*10¹³ VG/kg.

“Attach” or “Attached” or “Couple” or “Coupled” (and the like) means to chemically associate one entity (for example a moiety) with another. In some embodiments, the attaching is covalent, meaning that the attachment occurs in the context of the presence of a covalent bond between the two entities. In non-covalent embodiments, the non-covalent attaching is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. In embodiments, encapsulation is a form of attaching.

“Average”, as used herein, refers to the arithmetic mean unless otherwise noted.

“Concomitantly” means administering two or more materials/agents to a subject in a manner that is correlated in time, preferably sufficiently correlated in time so as to provide a modulation in an immune response, and even more preferably the two or more materials/agents are administered in combination. In embodiments, concomitant administration may encompass administration of two or more materials/agents within a specified period of time, preferably within 1 month, more preferably within 1 week, still more preferably within 1 day, and even more preferably within 1 hour. In embodiments, the materials/agents may be repeatedly administered concomitantly; that is concomitant administration on more than one occasion.

“Dose” refers to a specific quantity of a pharmacologically and/or immunologically active material for administration to a subject for a given time. In general, doses of the synthetic nanocarriers comprising an immunosuppressant and/or viral vectors in the methods and compositions of the invention refer to the amount of the synthetic nanocarriers comprising an immunosuppressant and/or viral vectors. Alternatively, the dose can be administered based on the number of synthetic nanocarriers that provide the desired amount of an immunosuppressant, in instances when referring to a dose of synthetic nanocarriers that comprise an immunosuppressant. When dose is used in the context of a repeated dosing, dose refers to the amount of each of the repeated doses, which may be the same or different.

“Encapsulate” means to enclose at least a portion of a substance within a synthetic nanocarrier. In some embodiments, a substance is enclosed completely within a synthetic nanocarrier. In other embodiments, most or all of a substance that is encapsulated is not exposed to the local environment external to the synthetic nanocarrier. In other embodiments, no more than 50%, 40%, 30%, 20%, 10% or 5% (weight/weight) is exposed to the local environment. Encapsulation is distinct from absorption, which places most or all of a substance on a surface of a synthetic nanocarrier, and leaves the substance exposed to the local environment external to the synthetic nanocarrier.

“Expression control sequences” are any sequences that can affect expression and can include promoters, enhancers, and operators. In one embodiment of any one of the methods or compositions provided, the expression control sequence is a promoter. In one embodiment of any one of the methods or compositions provided, the expression control sequence is a liver-specific promoter or a constitutive promoter. “Liver-specific promoters” are those that exclusively or preferentially result in expression in cells of the liver. “Constitutive promoters” are those that are thought of being generally active and not exclusive or preferential to certain cells. In any one of the nucleic acids or viral vectors provided herein the promoter may be any one of the promoters provided herein.

“Immunosuppressant” means a compound that can cause a tolerogenic effect, preferably through its effects on APCs. A tolerogenic effect generally refers to the modulation by the APC or other immune cells systemically and/or locally, that reduces, inhibits or prevents an undesired immune response to an antigen in a durable fashion. In one embodiment, the immunosuppressant is one that causes an APC to promote a regulatory phenotype in one or more immune effector cells. For example, the regulatory phenotype may be characterized by the inhibition of the production, induction, stimulation or recruitment of antigen-specific CD4+ T cells or B cells, the inhibition of the production of antigen-specific antibodies, the production, induction, stimulation or recruitment of Treg cells (e.g., CD4+CD25highFoxP3+Treg cells), etc. This may be the result of the conversion of CD4+ T cells or B cells to a regulatory phenotype. This may also be the result of induction of FoxP3 in other immune cells, such as CD8+ T cells, macrophages and iNKT cells. In one embodiment, the immunosuppressant is one that affects the response of the APC after it processes an antigen. In another embodiment, the immunosuppressant is not one that interferes with the processing of the antigen. In a further embodiment, the immunosuppressant is not an apoptotic-signaling molecule. In another embodiment, the immunosuppressant is not a phospholipid.

Immunosuppressants include, but are not limited to, statins; mTOR inhibitors, such as rapamycin or a rapamycin analog (i.e., rapalog); TGF-β signaling agents; TGF-β receptor agonists; histone deacetylase inhibitors, such as Trichostatin A; corticosteroids; inhibitors of mitochondrial function, such as rotenone; P38 inhibitors; NF-κβ inhibitors, such as 6Bio, Dexamethasone, TCPA-1, IKK VII; adenosine receptor agonists; prostaglandin E2 agonists (PGE2), such as Misoprostol; phosphodiesterase inhibitors, such as phosphodiesterase 4 inhibitor (PDE4), such as Rolipram; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors; PI3 KB inhibitors, such as TGX-221; autophagy inhibitors, such as 3-Methyladenine; aryl hydrocarbon receptor inhibitors; proteasome inhibitor I(PSI); and oxidized ATPs, such as P2X receptor blockers. Immunosuppressants also include IDO, vitamin D3, retinoic acid, cyclosporins, such as cyclosporine A, aryl hydrocarbon receptor inhibitors, resveratrol, azathiopurine (Aza), 6-mercaptopurine (6-MP), 6-thioguanine (6-TG), FK506, sanglifehrin A, salmeterol, mycophenolate mofetil (MMF), aspirin and other COX inhibitors, niflumic acid, estriol and triptolide. Other exemplary immunosuppressants include, but are not limited, small molecule drugs, natural products, antibodies (e.g., antibodies against CD20, CD3, CD4), biologics-based drugs, carbohydrate-based drugs, RNAi, antisense nucleic acids, aptamers, methotrexate, NSAIDs; fingolimod; natalizumab; alemtuzumab; anti-CD3; tacrolimus (FK506), abatacept, belatacept, etc. “Rapalog”, as used herein, refers to a molecule that is structurally related to (an analog) of rapamycin (sirolimus). Examples of rapalogs include, without limitation, temsirolimus (CCI-779), everolimus (RAD001), ridaforolimus (AP-23573), and zotarolimus (ABT-578). Additional examples of rapalogs may be found, for example, in WO Publication WO 1998/002441 and U.S. Pat. No. 8,455,510, the rapalogs of which are incorporated herein by reference in their entirety.

The immunosuppressant can be a compound that directly provides the tolerogenic effect on APCs or it can be a compound that provides the tolerogenic effect indirectly (i.e., after being processed in some way after administration). Further immunosuppressants, are known to those of skill in the art, and the invention is not limited in this respect. In embodiments, the immunosuppressant may comprise any one of the agents provided herein.

“Load”, when coupled to a synthetic nanocarrier, is the amount of the immunosuppressant coupled to the synthetic nanocarrier based on the total dry recipe weight of materials in an entire synthetic nanocarrier (weight/weight). Generally, such a load is calculated as an average across a population of synthetic nanocarriers. In one embodiment, the load on average across the synthetic nanocarriers is between 0.1% and 99%. In another embodiment, the load is between 0.1% and 50%. In another embodiment, the load is between 0.1% and 20%. In a further embodiment, the load is between 0.1% and 10%. In still a further embodiment, the load is between 1% and 10%. In still a further embodiment, the load is between 7% and 20%. In yet another embodiment, the load is at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 40%, at least 50%, at least 60%, at least 70%, at least 80%, at least 90%, at least 95%, at least 96%, at least 97%, at least 98% or at least 99% on average across the population of synthetic nanocarriers. In yet a further embodiment, the load is 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19% or 20% on average across the population of synthetic nanocarriers. In some embodiments of the above embodiments, the load is no more than 25% on average across a population of synthetic nanocarriers. In embodiments, the load is calculated as may be described in the Examples or as otherwise known in the art.

“Maximum dimension of a synthetic nanocarrier” means the largest dimension of a nanocarrier measured along any axis of the synthetic nanocarrier. “Minimum dimension of a synthetic nanocarrier” means the smallest dimension of a synthetic nanocarrier measured along any axis of the synthetic nanocarrier. For example, for a spheroidal synthetic nanocarrier, the maximum and minimum dimension of a synthetic nanocarrier would be substantially identical, and would be the size of its diameter. Similarly, for a cuboidal synthetic nanocarrier, the minimum dimension of a synthetic nanocarrier would be the smallest of its height, width or length, while the maximum dimension of a synthetic nanocarrier would be the largest of its height, width or length. In an embodiment, a minimum dimension of at least 75%, preferably at least 80%, more preferably at least 90%, of the synthetic nanocarriers in a sample, based on the total number of synthetic nanocarriers in the sample, is equal to or greater than 100 nm. In an embodiment, a maximum dimension of at least 75%, preferably at least 80%, more preferably at least 90%, of the synthetic nanocarriers in a sample, based on the total number of synthetic nanocarriers in the sample, is equal to or less than 5 μm. Preferably, a minimum dimension of at least 75%, preferably at least 80%, more preferably at least 90%, of the synthetic nanocarriers in a sample, based on the total number of synthetic nanocarriers in the sample, is greater than 110 nm, more preferably greater than 120 nm, more preferably greater than 130 nm, and more preferably still greater than 150 nm. Aspects ratios of the maximum and minimum dimensions of synthetic nanocarriers may vary depending on the embodiment. For instance, aspect ratios of the maximum to minimum dimensions of the synthetic nanocarriers may vary from 1:1 to 1,000,000:1, preferably from 1:1 to 100,000:1, more preferably from 1:1 to 10,000:1, more preferably from 1:1 to 1000:1, still more preferably from 1:1 to 100:1, and yet more preferably from 1:1 to 10:1.

Preferably, a maximum dimension of at least 75%, preferably at least 80%, more preferably at least 90%, of the synthetic nanocarriers in a sample, based on the total number of synthetic nanocarriers in the sample is equal to or less than 3 μm, more preferably equal to or less than 2 μm, more preferably equal to or less than 1 μm, more preferably equal to or less than 800 nm, more preferably equal to or less than 600 nm, and more preferably still equal to or less than 500 nm. In preferred embodiments, a minimum dimension of at least 75%, preferably at least 80%, more preferably at least 90%, of the synthetic nanocarriers in a sample, based on the total number of synthetic nanocarriers in the sample, is equal to or greater than 100 nm, more preferably equal to or greater than 120 nm, more preferably equal to or greater than 130 nm, more preferably equal to or greater than 140 nm, and more preferably still equal to or greater than 150 nm. Measurement of synthetic nanocarrier dimensions (e.g., effective diameter) may be obtained, in some embodiments, by suspending the synthetic nanocarriers in a liquid (usually aqueous) media and using dynamic light scattering (DLS) (e.g. using a Brookhaven ZetaPALS instrument). For example, a suspension of synthetic nanocarriers can be diluted from an aqueous buffer into purified water to achieve a final synthetic nanocarrier suspension concentration of approximately 0.01 to 0.1 mg/mL. The diluted suspension may be prepared directly inside, or transferred to, a suitable cuvette for DLS analysis. The cuvette may then be placed in the DLS, allowed to equilibrate to the controlled temperature, and then scanned for sufficient time to acquire a stable and reproducible distribution based on appropriate inputs for viscosity of the medium and refractive indicia of the sample. The effective diameter, or mean of the distribution, is then reported. Determining the effective sizes of high aspect ratio, or non-spheroidal, synthetic nanocarriers may require augmentative techniques, such as electron microscopy, to obtain more accurate measurements. “Dimension” or “size” or “diameter” of synthetic nanocarriers means the mean of a particle size distribution, for example, obtained using dynamic light scattering.

“Organic acidemia” refers to any disorder or defect whereby there is abnormal amino acid metabolism, such as with respect to branched-chain amino acids. Generally, this is caused by a mutation that results in such a deficiency in a subject. Thus, an “enzyme associated with the organic acidemia” is an enzyme in which there is a deficiency that results in the disorder in the subject. The four major types of organic acidemia are: methylmalonic acidemia (MMA), propionic acidemia, isovaleric acidemia, and maple syrup urine disease.

“Pharmaceutically acceptable excipient” or “pharmaceutically acceptable carrier” means a pharmacologically inactive material used together with a pharmacologically active material to formulate the compositions. Pharmaceutically acceptable excipients comprise a variety of materials known in the art, including but not limited to saccharides (such as glucose, lactose, and the like), preservatives such as antimicrobial agents, reconstitution aids, colorants, saline (such as phosphate buffered saline), and buffers.

“Repeat dose” or “repeat dosing” or the like means at least one additional dose or dosing that is administered to a subject subsequent to an earlier dose or dosing of the same material. For example, a repeated dose of a viral vector is at least one additional dose of the viral vector after a prior dose of the same material. While the material may be the same, the amount of the material in the repeated dose may be different from the earlier dose. For example, in an embodiment of any one of the methods or compositions provided herein, the amount of the viral vector in the repeated dose may be less than the amount of the viral vector of the earlier dose. Alternatively, in an embodiment of any one of the methods or compositions provided herein, the repeated dose may be in an amount that is at least equal to the amount of the viral vector in the earlier dose. A repeat dose may be administered weeks, months or years after the prior dose. In some embodiments of any one of the methods provided herein, the repeat dose or dosing is administered at least 1 week after the dose or dosing that occurred just prior to the repeat dose or dosing. In some embodiments of any one of the methods provided herein, the repeat dose or dosing is administered at least 1 month after the dose or dosing that occurred just prior to the repeat dose or dosing. Repeat dosing is considered to be efficacious if it results in a beneficial effect for the subject. Preferably, efficacious repeat dosing results in a beneficial effect in conjunction with reduced immune response, such as to the viral vector.

“Subject” means animals, including warm blooded mammals such as humans and primates; avians; domestic household or farm animals such as cats, dogs, sheep, goats, cattle, horses and pigs; laboratory animals such as mice, rats and guinea pigs; fish; reptiles; zoo and wild animals; and the like. As used herein, a subject may be in one need of any one of the methods or compositions provided herein. In some embodiments, the subject has or is suspected of having organic academia. In some embodiments, the subject is at risk of developing organic academia. In some embodiments, the organic academia is methylmalonic academia. In some embodiments, the organic academia is juvenile methylmalonic academia. In some embodiments, the subject is a pediatric or juvenile subject, e.g., is less than 18, less than 16, less than 15, less than 14, less than 13, less than 12, less than 11, less than 10, less than 9, less than 8, less than 7, less than 6, less than 5, less than 4, less than 3 year old, or less than 2 year old. In some embodiments, the subject is an adult subject. “Synthetic nanocarrier(s)” means a discrete object that is not found in nature, and that possesses at least one dimension that is less than or equal to 5 microns in size. Albumin nanoparticles are generally included as synthetic nanocarriers, however in certain embodiments the synthetic nanocarriers do not comprise albumin nanoparticles. In embodiments, synthetic nanocarriers do not comprise chitosan. In other embodiments, synthetic nanocarriers are not lipid-based nanoparticles. In further embodiments, synthetic nanocarriers do not comprise a phospholipid.

A synthetic nanocarrier can be, but is not limited to, one or a plurality of lipid-based nanoparticles (also referred to herein as lipid nanoparticles, i.e., nanoparticles where the majority of the material that makes up their structure are lipids), polymeric nanoparticles, metallic nanoparticles, surfactant-based emulsions, dendrimers, buckyballs, nanowires, virus-like particles (i.e., particles that are primarily made up of viral structural proteins but that are not infectious or have low infectivity), peptide or protein-based particles (also referred to herein as protein particles, i.e., particles where the majority of the material that makes up their structure are peptides or proteins) (such as albumin nanoparticles) and/or nanoparticles that are developed using a combination of nanomaterials such as lipid-polymer nanoparticles. Synthetic nanocarriers may be a variety of different shapes, including but not limited to spheroidal, cuboidal, pyramidal, oblong, cylindrical, toroidal, and the like. Synthetic nanocarriers according to the invention comprise one or more surfaces. Exemplary synthetic nanocarriers that can be adapted for use in the practice of the present invention comprise: (1) the biodegradable nanoparticles disclosed in U.S. Pat. No. 5,543,158 to Gref et al., (2) the polymeric nanoparticles of Published US Patent Application 20060002852 to Saltzman et al., (3) the lithographically constructed nanoparticles of Published US Patent Application 20090028910 to DeSimone et al., (4) the disclosure of WO 2009/051837 to von Andrian et al., (5) the nanoparticles disclosed in Published US Patent Application 2008/0145441 to Penades et al., (6) the protein nanoparticles disclosed in Published US Patent Application 20090226525 to de los Rios et al., (7) the virus-like particles disclosed in published US Patent Application 20060222652 to Sebbel et al., (8) the nucleic acid attached virus-like particles disclosed in published US Patent Application 20060251677 to Bachmann et al., (9) the virus-like particles disclosed in WO2010047839A1 or WO2009106999A2, (10) the nanoprecipitated nanoparticles disclosed in P. Paolicelli et al., “Surface-modified PLGA-based Nanoparticles that can Efficiently Associate and Deliver Virus-like Particles” Nanomedicine. 5(6):843-853 (2010), (11) apoptotic cells, apoptotic bodies or the synthetic or semisynthetic mimics disclosed in U.S. Publication 2002/0086049, or (12) those of Look et al., Nanogel-based delivery of mycophenolic acid ameliorates systemic lupus erythematosus in mice” J. Clinical Investigation 123(4):1741-1749(2013).

Synthetic nanocarriers according to the invention that have a minimum dimension of equal to or less than about 100 nm, preferably equal to or less than 100 nm, do not comprise a surface with hydroxyl groups that activate complement or alternatively comprise a surface that consists essentially of moieties that are not hydroxyl groups that activate complement. In a preferred embodiment, synthetic nanocarriers according to the invention that have a minimum dimension of equal to or less than about 100 nm, preferably equal to or less than 100 nm, do not comprise a surface that substantially activates complement or alternatively comprise a surface that consists essentially of moieties that do not substantially activate complement. In a more preferred embodiment, synthetic nanocarriers according to the invention that have a minimum dimension of equal to or less than about 100 nm, preferably equal to or less than 100 nm, do not comprise a surface that activates complement or alternatively comprise a surface that consists essentially of moieties that do not activate complement. In embodiments, synthetic nanocarriers exclude virus-like particles. In embodiments, synthetic nanocarriers may possess an aspect ratio greater than 1:1, 1:1.2, 1:1.5, 1:2, 1:3, 1:5, 1:7, or greater than 1:10.

“Viral vector” means a vector construct with viral components, such as capsid and/or coat proteins, that has been adapted to comprise and deliver a transgene or nucleic acid material that encodes therapeutic, such as a therapeutic protein, which transgene or nucleic acid material can be expressed as provided herein. “Expressed” or “expression” or the like refers to the synthesis of a functional (i.e., physiologically active for the desired purpose) product after the transgene or nucleic acid material is transduced into a cell and processed by the transduced cell. Such a product is also referred to herein as an “expression product”. Viral vectors can be based on, without limitation, adeno-associated viruses, such as AAV8 or AAV2. Viral vectors can also be based on Anc80. Thus, an AAV or Anc80 vector provided herein is a viral vector based on an AAV, such as AAV8 or AAV2, or Anc80, respectively, and has viral components, such as a capsid and/or coat protein, therefrom that can package for delivery the transgene or nucleic acid material. In some embodiments, the viral vector is a “chimeric viral vector”. In such embodiments, this means that the viral vector is made up of viral components that are derived from more than one virus or viral vector. Such a viral vector may be an AAV8/Anc80 or AAV2/Anc80 viral vector.

### C. Compositions for Use in the Inventive Methods

As mentioned above, there is no definitive treatment for methylmalonic acidemia (MMA). In addition, also as mentioned above, immune responses against a viral vector can adversely impact its efficacy and can also interfere with its readministration. Importantly, the methods and compositions provided herein have been found to overcome the aforementioned obstacles by achieving strong expression and/or reducing immune responses to viral vectors.

### Transgenes

The transgene or nucleic acid material, such as of the viral vectors, provided herein may encode any protein or portion thereof beneficial to a subject, such as one with a disease or disorder. Generally, the subject has or is suspected of having a disease or disorder whereby the subject's endogenous version of the protein is defective or produced in limited amounts or not at all. The subject may be one with any one of the diseases or disorders as provided herein, and the transgene or nucleic acid material is one that encodes any one of the therapeutic proteins or portion thereof as provided herein. The transgene or nucleic acid material provided herein may encode a functional version of any protein that through some defect in the endogenous version of which in a subject (including a defect in the expression of the endogenous version) results in a disease or disorder in the subject. Examples of such diseases or disorders include, but are not limited to, organic acidemias, such as methylmalonic acidemia (MMA). It follows that therapeutic proteins encoded by the transgene or nucleic acid material includes methylmalonyl-CoA mutase (MUT), including any wild-type version of MUT, an enzyme that is frequently mutated in cases of MMA.

The sequence of a transgene or nucleic acid material may also include an expression control sequence. Expression control sequences include promoters, enhancers, and operators, and are generally selected based on the expression systems in which the expression construct is to be utilized. In some embodiments, promoter and enhancer sequences are selected for the ability to increase gene expression, while operator sequences may be selected for the ability to regulate gene expression. The transgene may also include sequences that facilitate, and preferably promote, homologous recombination in a host cell. The transgene may also include sequences that are necessary for replication in a host cell.

Exemplary expression control sequences include liver-specific promoter sequences and constitutive promoter sequences, such as any one that may be provided herein. Generally, promoters are operatively linked upstream (i.e., 5′) of the sequence coding for a desired expression product. The transgene also may include a suitable polyadenylation sequence operably linked downstream (i.e., 3′) of the coding sequence.

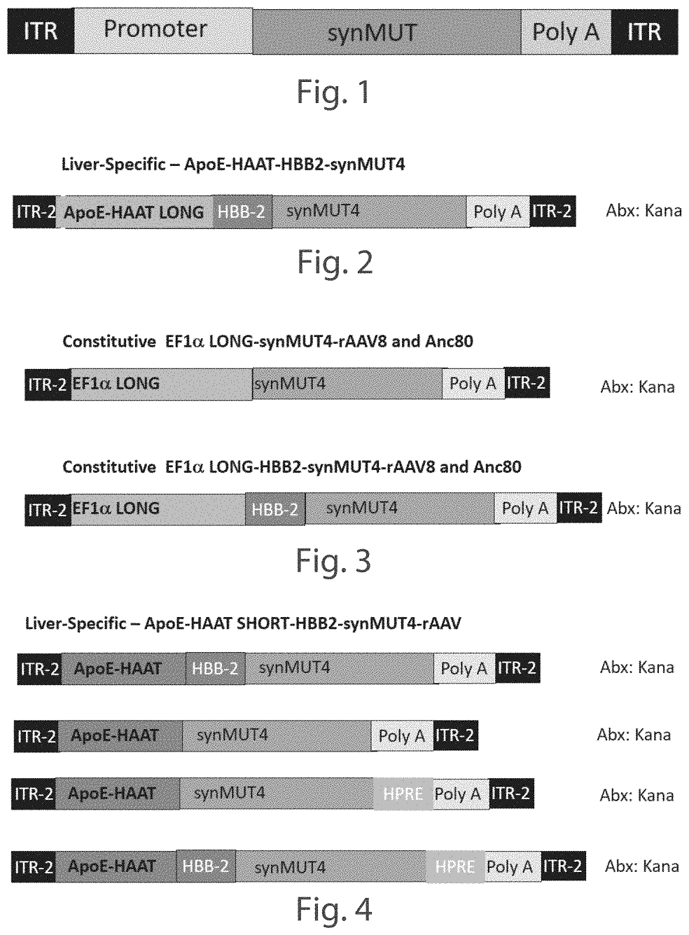

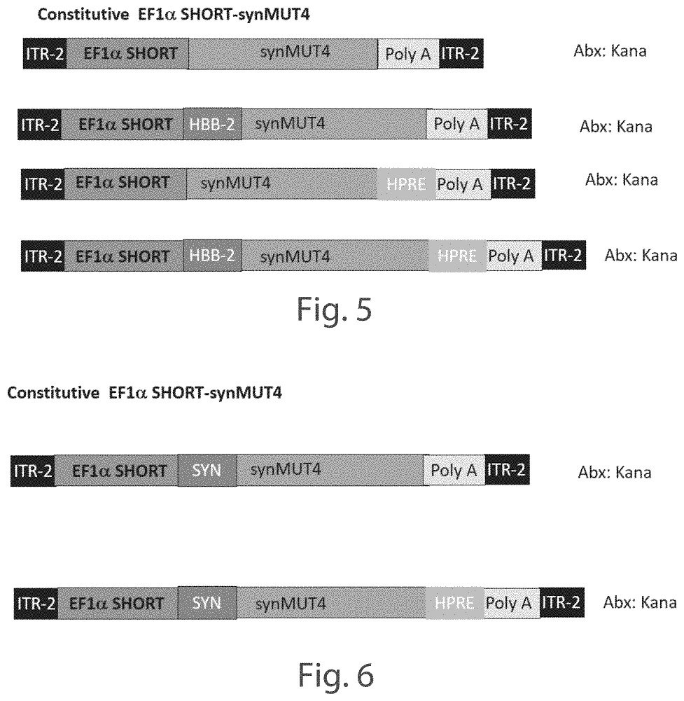

Exemplary constructs include those shown in the Figures. In some embodiments, the construct comprises an inverted terminal repeat (ITR), a promoter (for example, a liver-specific promoter or a constitutive promoter), synthetic methyl malonyl CoA mutase 4 (synMUT), a poly A tail, and an ITR, as shown in FIG. 1. In some embodiments, the promoter is a constitutive promoter, such as elongation factor 1 alpha 1 (FIGS. 3, 5). In other embodiments, the promoter is a liver-specific promoter, such as apolipoprotein E-human alpha-1-antitrypsin (apoE-hAAT) (FIGS. 2, 4). In some embodiments, the promoter and the synMUT4 segment may be separated by an intron, for example hemoglobin subunit beta-2 (HBB-2), as illustrated in FIGS. 2-5. In other embodiments, the promoter and the synMUT4 segment may be separated by a synthetic (SYN) intron, as depicted in FIG. 6. In some embodiments, there is no intron separating the promoter and the synMUT4 segment. In some embodiments, the synMUT4 is followed by a post-transcriptional regulatory sequence, such as a human post-transcriptional regulatory element (FIGS. 4-6).

Nucleic acids that encode a MUT or a portion thereof, are provided in one aspect. Compositions of such nucleic acids are also provided. The nucleic acids may include any one of the types of specific promoters provided herein and encode a MUT or portion thereof. Any one of the nucleic acids provided may include any one of the ITRs and/or a poly A tail as provided herein. Any one of the nucleic acids provided herein may include any one of the introns provided herein, such as between the promoter and sequence encoding a MUT or portion thereof. Any one of the nucleic acids provided herein may include any one of the post-transcriptional regulatory sequences provided herein, such as following the sequence encoding a MUT or portion thereof. A viral vector comprising any one of the nucleic acids provided herein is provided in one aspect. Such a viral vector may be an AAV vector, such as an AAV8 or AAV2 vector, or an Anc80 vector or an AAV8/Anc80 or an AAV2/Anc80 vector. Any one of the nucleic acids or vectors provided herein may be for use in any one of the methods provided herein.

### Viral Vectors

Viruses have evolved specialized mechanisms to transport their genomes inside the cells that they infect; viral vectors based on such viruses can be tailored to transduce cells to specific applications. Examples of viral vectors that may be used as provided herein are known in the art or described herein. Suitable viral vectors include, for instance, adeno-associated virus (AAV)-based vectors.

The viral vectors provided herein can be based on adeno-associated viruses (AAVs). AAV vectors have been of particular interest for use in therapeutic applications such as those described herein. AAV is a DNA virus, which is not known to cause human disease. Generally, AAV requires co-infection with a helper virus (e.g., an adenovirus or a herpes virus), or expression of helper genes, for efficient replication. For a description of AAV-based vectors, see, for example, U.S. Pat. Nos. 8,679,837, 8,637,255, 8,409,842, 7,803,622, and 7,790,449, and U.S. Publication Nos. 20150065562, 20140155469, 20140037585, 20130096182, 20120100606, and 20070036757. The AAV vectors may be recombinant AAV vectors. The AAV vectors may also be self-complementary (sc) AAV vectors, which are described, for example, in U.S. Patent Publications 2007/01110724 and 2004/0029106, and U.S. Pat. Nos. 7,465,583 and 7,186,699.

The adeno-associated virus on which a viral vector is based may be of a specific serotype, such as AAV8 or AAV2. In some embodiments of any one of the methods or compositions provided herein, therefore, the AAV vector is an AAV8 or AAV2 vector.

In some embodiments, the virus on which a viral vector is based may be synthetic, such as Anc80.

In some embodiments, the viral vector is an AAV/Anc80 vectors, such as an AAV8/Anc80 vector or an AAV2/Anc80 vector.

### Synthetic Nanocarriers Comprising an Immunosuppressant

The viral vectors provided herein can be administered in combination with synthetic nanocarriers comprising an immunosuppressant. Generally, the immunosuppressant is an element that is in addition to the material that makes up the structure of the synthetic nanocarrier. For example, in one embodiment, where the synthetic nanocarrier is made up of one or more polymers, the immunosuppressant is a compound that is in addition and, in some embodiments, attached to the one or more polymers. In embodiments where the material of the synthetic nanocarrier also results in a tolerogenic effect, the immunosuppressant is an element present in addition to the material of the synthetic nanocarrier that results in a tolerogenic effect.

A wide variety of other synthetic nanocarriers can be used according to the invention, and in some embodiments, coupled to an immunosuppressant. In some embodiments, synthetic nanocarriers are spheres or spheroids. In some embodiments, synthetic nanocarriers are flat or plate-shaped. In some embodiments, synthetic nanocarriers are cubes or cubic. In some embodiments, synthetic nanocarriers are ovals or ellipses. In some embodiments, synthetic nanocarriers are cylinders, cones, or pyramids.

In some embodiments, it is desirable to use a population of synthetic nanocarriers that is relatively uniform in terms of size or shape so that each synthetic nanocarrier has similar properties. For example, at least 80%, at least 90%, or at least 95% of the synthetic nanocarriers of any one of the compositions or methods provided, based on the total number of synthetic nanocarriers, may have a minimum dimension or maximum dimension that falls within 5%, 10%, or 20% of the average diameter or average dimension of the synthetic nanocarriers.

Synthetic nanocarriers can be solid or hollow and can comprise one or more layers. In some embodiments, each layer has a unique composition and unique properties relative to the other layer(s). To give but one example, synthetic nanocarriers may have a core/shell structure, wherein the core is one layer (e.g. a polymeric core) and the shell is a second layer (e.g. a lipid bilayer or monolayer). Synthetic nanocarriers may comprise a plurality of different layers.

In some embodiments, synthetic nanocarriers may optionally comprise one or more lipids. In some embodiments, a synthetic nanocarrier may comprise a liposome. In some embodiments, a synthetic nanocarrier may comprise a lipid bilayer. In some embodiments, a synthetic nanocarrier may comprise a lipid monolayer. In some embodiments, a synthetic nanocarrier may comprise a micelle. In some embodiments, a synthetic nanocarrier may comprise a core comprising a polymeric matrix surrounded by a lipid layer (e.g., lipid bilayer, lipid monolayer, etc.). In some embodiments, a synthetic nanocarrier may comprise a non-polymeric core (e.g., metal particle, quantum dot, ceramic particle, bone particle, viral particle, proteins, nucleic acids, carbohydrates, etc.) surrounded by a lipid layer (e.g., lipid bilayer, lipid monolayer, etc.).

In other embodiments, synthetic nanocarriers may comprise metal particles, quantum dots, ceramic particles, etc. In some embodiments, a non-polymeric synthetic nanocarrier is an aggregate of non-polymeric components, such as an aggregate of metal atoms (e.g., gold atoms).

In some embodiments, synthetic nanocarriers may optionally comprise one or more amphiphilic entities. In some embodiments, an amphiphilic entity can promote the production of synthetic nanocarriers with increased stability, improved uniformity, or increased viscosity. In some embodiments, amphiphilic entities can be associated with the interior surface of a lipid membrane (e.g., lipid bilayer, lipid monolayer, etc.). Many amphiphilic entities known in the art are suitable for use in making synthetic nanocarriers in accordance with the present invention. Such amphiphilic entities include, but are not limited to, phosphoglycerides; phosphatidylcholines; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohols such as polyethylene glycol (PEG); polyoxyethylene-9-lauryl ether; a surface active fatty acid, such as palmitic acid or oleic acid; fatty acids; fatty acid monoglycerides; fatty acid diglycerides; fatty acid amides; sorbitan trioleate (Span®85) glycocholate; sorbitan monolaurate (Span®20); polysorbate 20 (Tween®20); polysorbate 60 (Tween®60); polysorbate 65 (Tween®65); polysorbate 80 (Tween®80); polysorbate 85 (Tween®85); polyoxyethylene monostearate; surfactin; a poloxomer; a sorbitan fatty acid ester such as sorbitan trioleate; lecithin; lysolecithin; phosphatidylserine; phosphatidylinositol;sphingomyelin; phosphatidylethanolamine (cephalin); cardiolipin; phosphatidic acid; cerebrosides; dicetylphosphate; dipalmitoylphosphatidylglycerol; stearylamine; dodecylamine; hexadecyl-amine; acetyl palmitate; glycerol ricinoleate; hexadecyl sterate; isopropyl myristate; tyloxapol; poly(ethylene glycol)5000-phosphatidylethanolamine; poly(ethylene glycol)400-monostearate; phospholipids; synthetic and/or natural detergents having high surfactant properties; deoxycholates; cyclodextrins; chaotropic salts; ion pairing agents; and combinations thereof. An amphiphilic entity component may be a mixture of different amphiphilic entities. Those skilled in the art will recognize that this is an exemplary, not comprehensive, list of substances with surfactant activity. Any amphiphilic entity may be used in the production of synthetic nanocarriers to be used in accordance with the present invention.

In some embodiments, synthetic nanocarriers may optionally comprise one or more carbohydrates. Carbohydrates may be natural or synthetic. A carbohydrate may be a derivatized natural carbohydrate. In certain embodiments, a carbohydrate comprises monosaccharide or disaccharide, including but not limited to glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid. In certain embodiments, a carbohydrate is a polysaccharide, including but not limited to pullulan, cellulose, microcrystalline cellulose, hydroxypropyl methylcellulose (HPMC), hydroxycellulose (HC), methylcellulose (MC), dextran, cyclodextran, glycogen, hydroxyethylstarch, carageenan, glycon, amylose, chitosan, N,O-carboxylmethylchitosan, algin and alginic acid, starch, chitin, inulin, konjac, glucommannan, pustulan, heparin, hyaluronic acid, curdlan, and xanthan. In embodiments, the synthetic nanocarriers do not comprise (or specifically exclude) carbohydrates, such as a polysaccharide. In certain embodiments, the carbohydrate may comprise a carbohydrate derivative such as a sugar alcohol, including but not limited to mannitol, sorbitol, xylitol, erythritol, maltitol, and lactitol.

In some embodiments, synthetic nanocarriers can comprise one or more polymers. In some embodiments, the synthetic nanocarriers comprise one or more polymers that is a non-methoxy-terminated, pluronic polymer. In some embodiments, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the synthetic nanocarriers are non-methoxy-terminated, pluronic polymers. In some embodiments, all of the polymers that make up the synthetic nanocarriers are non-methoxy-terminated, pluronic polymers. In some embodiments, the synthetic nanocarriers comprise one or more polymers that is a non-methoxy-terminated polymer. In some embodiments, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the synthetic nanocarriers are non-methoxy-terminated polymers. In some embodiments, all of the polymers that make up the synthetic nanocarriers are non-methoxy-terminated polymers. In some embodiments, the synthetic nanocarriers comprise one or more polymers that do not comprise pluronic polymer. In some embodiments, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the synthetic nanocarriers do not comprise pluronic polymer. In some embodiments, all of the polymers that make up the synthetic nanocarriers do not comprise pluronic polymer. In some embodiments, such a polymer can be surrounded by a coating layer (e.g., liposome, lipid monolayer, micelle, etc.). In some embodiments, elements of the synthetic nanocarriers can be attached to the polymer.

Immunosuppressants can be coupled to the synthetic nanocarriers by any of a number of methods. Generally, the attaching can be a result of bonding between the immunosuppressants and the synthetic nanocarriers. This bonding can result in the immunosuppressants being attached to the surface of the synthetic nanocarriers and/or contained (encapsulated) within the synthetic nanocarriers. In some embodiments, however, the immunosuppressants are encapsulated by the synthetic nanocarriers as a result of the structure of the synthetic nanocarriers rather than bonding to the synthetic nanocarriers. In preferable embodiments, the synthetic nanocarrier comprises a polymer as provided herein, and the immunosuppressants are attached to the polymer.

When attaching occurs as a result of bonding between the immunosuppressants and synthetic nanocarriers, the attaching may occur via a coupling moiety. A coupling moiety can be any moiety through which an immunosuppressant is bonded to a synthetic nanocarrier. Such moieties include covalent bonds, such as an amide bond or ester bond, as well as separate molecules that bond (covalently or non-covalently) the immunosuppressant to the synthetic nanocarrier. Such molecules include linkers or polymers or a unit thereof. For example, the coupling moiety can comprise a charged polymer to which an immunosuppressant electrostatically binds. As another example, the coupling moiety can comprise a polymer or unit thereof to which it is covalently bonded.

In preferred embodiments, the synthetic nanocarriers comprise a polymer as provided herein. These synthetic nanocarriers can be completely polymeric or they can be a mix of polymers and other materials.

In some embodiments, the polymers of a synthetic nanocarrier associate to form a polymeric matrix. In some of these embodiments, a component, such as an immunosuppressant, can be covalently associated with one or more polymers of the polymeric matrix. In some embodiments, covalent association is mediated by a linker. In some embodiments, a component can be noncovalently associated with one or more polymers of the polymeric matrix. For example, in some embodiments, a component can be encapsulated within, surrounded by, and/or dispersed throughout a polymeric matrix. Alternatively or additionally, a component can be associated with one or more polymers of a polymeric matrix by hydrophobic interactions, charge interactions, van der Waals forces, etc. A wide variety of polymers and methods for forming polymeric matrices therefrom are known conventionally.

Polymers may be natural or unnatural (synthetic) polymers. Polymers may be homopolymers or copolymers comprising two or more monomers. In terms of sequence, copolymers may be random, block, or comprise a combination of random and block sequences. Typically, polymers in accordance with the present invention are organic polymers.

In some embodiments, the polymer comprises a polyester, polycarbonate, polyamide, or polyether, or unit thereof. In other embodiments, the polymer comprises poly(ethylene glycol) (PEG), polypropylene glycol, poly(lactic acid), poly(glycolic acid), poly(lactic-co-glycolic acid), or a polycaprolactone, or unit thereof. In some embodiments, it is preferred that the polymer is biodegradable. Therefore, in these embodiments, it is preferred that if the polymer comprises a polyether, such as poly(ethylene glycol) or polypropylene glycol or unit thereof, the polymer comprises a block-co-polymer of a polyether and a biodegradable polymer such that the polymer is biodegradable. In other embodiments, the polymer does not solely comprise a polyether or unit thereof, such as poly(ethylene glycol) or polypropylene glycol or unit thereof.

Other examples of polymers suitable for use in the present invention include, but are not limited to polyethylenes, polycarbonates (e.g. poly(1,3-dioxan-2one)), polyanhydrides (e.g. poly(sebacic anhydride)), polypropylfumerates, polyamides (e.g. polycaprolactam), polyacetals, polyethers, polyesters (e.g., polylactide, polyglycolide, polylactide-co-glycolide, polycaprolactone, polyhydroxyacid (e.g. poly(β-hydroxyalkanoate))), poly(orthoesters), polycyanoacrylates, polyvinyl alcohols, polyurethanes, polyphosphazenes, polyacrylates, polymethacrylates, polyureas, polystyrenes, and polyamines, polylysine, polylysine-PEG copolymers, and poly(ethyleneimine), poly(ethylene imine)-PEG copolymers.

In some embodiments, polymers in accordance with the present invention include polymers which have been approved for use in humans by the U.S. Food and Drug Administration (FDA) under 21 C.F.R. § 177.2600, including but not limited to polyesters (e.g., polylactic acid, poly(lactic-co-glycolic acid), polycaprolactone, polyvalerolactone, poly(1,3-dioxan-2one)); polyanhydrides (e.g., poly(sebacic anhydride)); polyethers (e.g., polyethylene glycol); polyurethanes; polymethacrylates; polyacrylates; and polycyanoacrylates.

In some embodiments, polymers can be hydrophilic. For example, polymers may comprise anionic groups (e.g., phosphate group, sulphate group, carboxylate group); cationic groups (e.g., quaternary amine group); or polar groups (e.g., hydroxyl group, thiol group, amine group). In some embodiments, a synthetic nanocarrier comprising a hydrophilic polymeric matrix generates a hydrophilic environment within the synthetic nanocarrier. In some embodiments, polymers can be hydrophobic. In some embodiments, a synthetic nanocarrier comprising a hydrophobic polymeric matrix generates a hydrophobic environment within the synthetic nanocarrier. Selection of the hydrophilicity or hydrophobicity of the polymer may have an impact on the nature of materials that are incorporated within the synthetic nanocarrier.

In some embodiments, polymers may be modified with one or more moieties and/or functional groups. A variety of moieties or functional groups can be used in accordance with the present invention. In some embodiments, polymers may be modified with polyethylene glycol (PEG), with a carbohydrate, and/or with acyclic polyacetals derived from polysaccharides (Papisov, 2001, ACS Symposium Series, 786:301). Certain embodiments may be made using the general teachings of U.S. Pat. No. 5,543,158 to Gref et al., or WO publication WO2009/051837 by von Andrian et al.

In some embodiments, polymers may be modified with a lipid or fatty acid group. In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

In some embodiments, polymers may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEG copolymers and copolymers of lactide and glycolide (e.g., PLA-PEG copolymers, PGA-PEG copolymers, PLGA-PEG copolymers, and derivatives thereof. In some embodiments, polyesters include, for example, poly(caprolactone), poly(caprolactone)-PEG copolymers, poly(L-lactide-co-L-lysine), poly(serine ester), poly(4-hydroxy-L-proline ester), poly[α-(4-aminobutyl)-L-glycolic acid], and derivatives thereof.

In some embodiments, a polymer may be PLGA. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid:glycolic acid ratio. In some embodiments, PLGA to be used in accordance with the present invention is characterized by a lactic acid:glycolic acid ratio of approximately 85:15, approximately 75:25, approximately 60:40, approximately 50:50, approximately 40:60, approximately 25:75, or approximately 15:85.

In some embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

In some embodiments, polymers can be cationic polymers. In general, cationic polymers are able to condense and/or protect negatively charged strands of nucleic acids. Amine-containing polymers such as poly(lysine) (Zauner et al., 1998, Adv. Drug Del. Rev., 30:97; and Kabanov et al., 1995, Bioconjugate Chem., 6:7), poly(ethylene imine) (PEI; Boussif et al., 1995, Proc. Natl. Acad. Sci., USA, 1995, 92:7297), and poly(amidoamine) dendrimers (Kukowska-Latallo et al., 1996, Proc. Natl. Acad. Sci., USA, 93:4897; Tang et al., 1996, Bioconjugate Chem., 7:703; and Haensler et al., 1993, Bioconjugate Chem., 4:372) are positively-charged at physiological pH, form ion pairs with nucleic acids. In embodiments, the synthetic nanocarriers may not comprise (or may exclude) cationic polymers.

In some embodiments, polymers can be degradable polyesters bearing cationic side chains (Putnam et al., 1999, Macromolecules, 32:3658; Barrera et al., 1993, J. Am. Chem. Soc., 115:11010; Kwon et al., 1989, Macromolecules, 22:3250; Lim et al., 1999, J. Am. Chem. Soc., 121:5633; and Zhou et al., 1990, Macromolecules, 23:3399). Examples of these polyesters include poly(L-lactide-co-L-lysine) (Barrera et al., 1993, J. Am. Chem. Soc., 115:11010), poly(serine ester) (Zhou et al., 1990, Macromolecules, 23:3399), poly(4-hydroxy-L-proline ester) (Putnam et al., 1999, Macromolecules, 32:3658; and Lim et al., 1999, J. Am. Chem. Soc., 121:5633), and poly(4-hydroxy-L-proline ester) (Putnam et al., 1999, Macromolecules, 32:3658; and Lim et al., 1999, J. Am. Chem. Soc., 121:5633).

The properties of these and other polymers and methods for preparing them are well known in the art (see, for example, U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and U.S. Pat. No. 4,946,929; Wang et al., 2001, J. Am. Chem. Soc., 123:9480; Lim et al., 2001, J. Am. Chem. Soc., 123:2460; Langer, 2000, Acc. Chem. Res., 33:94; Langer, 1999, J. Control. Release, 62:7; and Uhrich et al., 1999, Chem. Rev., 99:3181). More generally, a variety of methods for synthesizing certain suitable polymers are described in Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., 1997, Nature, 390:386; and in U.S. Pat. Nos. 6,506,577, 6,632,922, 6,686,446, and 6,818,732.

In some embodiments, polymers can be linear or branched polymers. In some embodiments, polymers can be dendrimers. In some embodiments, polymers can be substantially cross-linked to one another. In some embodiments, polymers can be substantially free of cross-links. In some embodiments, polymers can be used in accordance with the present invention without undergoing a cross-linking step. It is further to be understood that the synthetic nanocarriers may comprise block copolymers, graft copolymers, blends, mixtures, and/or adducts of any of the foregoing and other polymers. Those skilled in the art will recognize that the polymers listed herein represent an exemplary, not comprehensive, list of polymers that can be of use in accordance with the present invention.

In some embodiments, synthetic nanocarriers do not comprise a polymeric component. In some embodiments, synthetic nanocarriers may comprise metal particles, quantum dots, ceramic particles, etc. In some embodiments, a non-polymeric synthetic nanocarrier is an aggregate of non-polymeric components, such as an aggregate of metal atoms (e.g., gold atoms).

Any immunosuppressant as provided herein can be, in some embodiments, coupled to synthetic nanocarriers. Immunosuppressants include, but are not limited to, statins; mTOR inhibitors, such as rapamycin or a rapamycin analog (rapalog); TGF-β signaling agents; TGF-β receptor agonists; histone deacetylase (HDAC) inhibitors; corticosteroids; inhibitors of mitochondrial function, such as rotenone; P38 inhibitors; NF-κβ inhibitors; adenosine receptor agonists; prostaglandin E2 agonists; phosphodiesterase inhibitors, such as phosphodiesterase 4 inhibitor; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors and oxidized ATPs. Immunosuppressants also include IDO, vitamin D3, cyclosporine A, aryl hydrocarbon receptor inhibitors, resveratrol, azathiopurine, 6-mercaptopurine, aspirin, niflumic acid, estriol, tripolide, interleukins (e.g., IL-1, IL-10), cyclosporine A, siRNAs targeting cytokines or cytokine receptors and the like.

Examples of mTOR inhibitors include rapamycin and analogs thereof (e.g., CCL-779, RAD001, AP23573, C20-methallylrapamycin (C20-Marap), C16-(S)-butylsulfonamidorapamycin (C16-BSrap), C16-(S)-3-methylindolerapamycin (C16-iRap) (Bayle et al. Chemistry & Biology 2006, 13:99-107)), AZD8055, BEZ235 (NVP-BEZ235), chrysophanic acid (chrysophanol), deforolimus (MK-8669), everolimus (RAD0001), KU-0063794, PI-103, PP242, temsirolimus, and WYE-354 (available from Selleck, Houston, Tex., USA).

Compositions according to the invention can comprise pharmaceutically acceptable excipients, such as preservatives, buffers, saline, or phosphate buffered saline. The compositions may be made using conventional pharmaceutical manufacturing and compounding techniques to arrive at useful dosage forms. In an embodiment, compositions are suspended in sterile saline solution for injection together with a preservative.

### D. Methods of Using and Making the Compositions and Related Methods

Viral vectors can be made with methods known to those of ordinary skill in the art or as otherwise described herein. For example, viral vectors can be constructed and/or purified using the methods set forth, for example, in U.S. Pat. No. 4,797,368 and Laughlin et al., Gene, 23, 65-73 (1983).

Viral vectors, such as AAV vectors, may be produced using recombinant methods. For example, the methods can involve culturing a host cell which contains a nucleic acid sequence encoding an AAV capsid protein or fragment thereof; a functional rep gene; a recombinant AAV vector composed of AAV inverted terminal repeats (ITRs) and a transgene; and sufficient helper functions to permit packaging of the recombinant AAV vector into the AAV capsid proteins.

The components to be cultured in the host cell to package a viral vector in a capsid may be provided to the host cell in trans. Alternatively, any one or more of the required components (e.g., recombinant viral vector, rep sequences, cap sequences, and/or helper functions) may be provided by a stable host cell which has been engineered to contain one or more of the required components using methods known to those of skill in the art. Most suitably, such a stable host cell can contain the required component(s) under the control of an inducible promoter. However, the required component(s) may be under the control of a constitutive promoter. The recombinant viral vector, rep sequences, cap sequences, and helper functions for producing the viral vector may be delivered to the packaging host cell using any appropriate genetic element. The selected genetic element may be delivered by any suitable method, including those described herein. The methods used to construct any embodiment of this invention are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present invention. See, e.g., K. Fisher et al, J. Virol., 70:520-532 (1993) and U.S. Pat. No. 5,478,745.

In some embodiments, recombinant AAV vectors may be produced using the triple transfection method (e.g., as described in detail in U.S. Pat. No. 6,001,650, the contents of which relating to the triple transfection method are incorporated herein by reference). Typically, the recombinant AAVs are produced by transfecting a host cell with a recombinant AAV vector (comprising a transgene) to be packaged into AAV particles, an AAV helper function vector, and an accessory function vector. Generally, an AAV helper function vector encodes AAV helper function sequences (rep and cap), which function in trans for productive AAV replication and encapsulation. Preferably, the AAV helper function vector supports efficient AAV vector production without generating any detectable wild-type AAV virions (i.e., AAV virions containing functional rep and cap genes). The accessory function vector can encode nucleotide sequences for non-AAV derived viral and/or cellular functions upon which AAV is dependent for replication. The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

Other methods for producing viral vectors are known in the art. Moreover, viral vectors are available commercially.

In regard to synthetic nanocarriers coupled to immunosuppressants, methods for attaching components to synthetic nanocarriers may be useful.

In certain embodiments, the attaching can be via a covalent linker. In embodiments, immunosuppressants according to the invention can be covalently attached to the external surface via a 1,2,3-triazole linker formed by the 1,3-dipolar cycloaddition reaction of azido groups with immunosuppressant containing an alkyne group or by the 1,3-dipolar cycloaddition reaction of alkynes with immunosuppressants containing an azido group. Such cycloaddition reactions are preferably performed in the presence of a Cu(I) catalyst along with a suitable Cu(I)-ligand and a reducing agent to reduce Cu(II) compound to catalytic active Cu(I) compound. This Cu(I)-catalyzed azide-alkyne cycloaddition (CuAAC) can also be referred as the click reaction.

Additionally, covalent coupling may comprise a covalent linker that comprises an amide linker, a disulfide linker, a thioether linker, a hydrazone linker, a hydrazide linker, an imine or oxime linker, an urea or thiourea linker, an amidine linker, an amine linker, or a sulfonamide linker.

An amide linker is formed via an amide bond between an amine on one component such as an immunosuppressant with the carboxylic acid group of a second component such as the nanocarrier. The amide bond in the linker can be made using any of the conventional amide bond forming reactions with suitably protected amino acids and activated carboxylic acid such N-hydroxysuccinimide-activated ester.

A disulfide linker is made via the formation of a disulfide (S—S) bond between two sulfur atoms of the form, for instance, of R1-S—S—R2. A disulfide bond can be formed by thiol exchange of a component containing thiol/mercaptan group(—SH) with another activated thiol group or a component containing thiol/mercaptan groups with a component containing activated thiol group.

A triazole linker, specifically a 1,2,3-triazole of the form

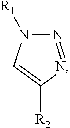

wherein R1 and R2 may be any chemical entities, is made by the 1,3-dipolar cycloaddition reaction of an azide attached to a first component with a terminal alkyne attached to a second component such as the immunosuppressant. The 1,3-dipolar cycloaddition reaction is performed with or without a catalyst, preferably with Cu(I)-catalyst, which links the two components through a 1,2,3-triazole function. This chemistry is described in detail by Sharpless et al., Angew. Chem. Int. Ed. 41(14), 2596, (2002) and Meldal, et al, Chem. Rev., 2008, 108(8), 2952-3015 and is often referred to as a “click” reaction or CuAAC.

A thioether linker is made by the formation of a sulfur-carbon (thioether) bond in the form, for instance, of R1-S—R2. Thioether can be made by either alkylation of a thiol/mercaptan (—SH) group on one component with an alkylating group such as halide or epoxide on a second component. Thioether linkers can also be formed by Michael addition of a thiol/mercaptan group on one component to an electron-deficient alkene group on a second component containing a maleimide group or vinyl sulfone group as the Michael acceptor. In another way, thioether linkers can be prepared by the radical thiol-ene reaction of a thiol/mercaptan group on one component with an alkene group on a second component.

A hydrazone linker is made by the reaction of a hydrazide group on one component with an aldehyde/ketone group on the second component.

A hydrazide linker is formed by the reaction of a hydrazine group on one component with a carboxylic acid group on the second component. Such reaction is generally performed using chemistry similar to the formation of amide bond where the carboxylic acid is activated with an activating reagent.

An imine or oxime linker is formed by the reaction of an amine or N-alkoxyamine (or aminooxy) group on one component with an aldehyde or ketone group on the second component.

An urea or thiourea linker is prepared by the reaction of an amine group on one component with an isocyanate or thioisocyanate group on the second component.

An amidine linker is prepared by the reaction of an amine group on one component with an imidoester group on the second component.

An amine linker is made by the alkylation reaction of an amine group on one component with an alkylating group such as halide, epoxide, or sulfonate ester group on the second component. Alternatively, an amine linker can also be made by reductive amination of an amine group on one component with an aldehyde or ketone group on the second component with a suitable reducing reagent such as sodium cyanoborohydride or sodium triacetoxyborohydride.

A sulfonamide linker is made by the reaction of an amine group on one component with a sulfonyl halide (such as sulfonyl chloride) group on the second component.

A sulfone linker is made by Michael addition of a nucleophile to a vinyl sulfone. Either the vinyl sulfone or the nucleophile may be on the surface of the nanocarrier or attached to a component.

The component can also be conjugated via non-covalent conjugation methods. For example, a negative charged immunosuppressant can be conjugated to a positive charged component through electrostatic adsorption. A component containing a metal ligand can also be conjugated to a metal complex via a metal-ligand complex.

In embodiments, the component can be attached to a polymer, for example polylactic acid-block-polyethylene glycol, prior to the assembly of a synthetic nanocarrier or the synthetic nanocarrier can be formed with reactive or activatible groups on its surface. In the latter case, the component may be prepared with a group which is compatible with the attachment chemistry that is presented by the synthetic nanocarriers' surface. In other embodiments, a peptide component can be attached to VLPs or liposomes using a suitable linker. A linker is a compound or reagent that capable of coupling two molecules together. In an embodiment, the linker can be a homobifuntional or heterobifunctional reagent as described in Hermanson 2008. For example, a VLP or liposome synthetic nanocarrier containing a carboxylic group on the surface can be treated with a homobifunctional linker, adipic dihydrazide (ADH), in the presence of EDC to form the corresponding synthetic nanocarrier with the ADH linker. The resulting ADH linked synthetic nanocarrier is then conjugated with a peptide component containing an acid group via the other end of the ADH linker on nanocarrier to produce the corresponding VLP or liposome peptide conjugate.

In embodiments, a polymer containing an azide or alkyne group, terminal to the polymer chain is prepared. This polymer is then used to prepare a synthetic nanocarrier in such a manner that a plurality of the alkyne or azide groups are positioned on the surface of that nanocarrier. Alternatively, the synthetic nanocarrier can be prepared by another route, and subsequently functionalized with alkyne or azide groups. The component is prepared with the presence of either an alkyne (if the polymer contains an azide) or an azide (if the polymer contains an alkyne) group. The component is then allowed to react with the nanocarrier via the 1,3-dipolar cycloaddition reaction with or without a catalyst which covalently attaches the component to the particle through the 1,4-disubstituted 1,2,3-triazole linker.

If the component is a small molecule it may be of advantage to attach the component to a polymer prior to the assembly of synthetic nanocarriers. In embodiments, it may also be an advantage to prepare the synthetic nanocarriers with surface groups that are used to attach the component to the synthetic nanocarrier through the use of these surface groups rather than attaching the component to a polymer and then using this polymer conjugate in the construction of synthetic nanocarriers.

For detailed descriptions of available conjugation methods, see Hermanson G T “Bioconjugate Techniques”, 2nd Edition Published by Academic Press, Inc., 2008. In addition to covalent attachment the component can be attached by adsorption to a pre-formed synthetic nanocarrier or it can be attached by encapsulation during the formation of the synthetic nanocarrier.

Synthetic nanocarriers may be prepared using a wide variety of methods known in the art. For example, synthetic nanocarriers can be formed by methods such as nanoprecipitation, flow focusing using fluidic channels, spray drying, single and double emulsion solvent evaporation, solvent extraction, phase separation, milling, microemulsion procedures, microfabrication, nanofabrication, sacrificial layers, simple and complex coacervation, and other methods well known to those of ordinary skill in the art. Alternatively or additionally, aqueous and organic solvent syntheses for monodisperse semiconductor, conductive, magnetic, organic, and other nanomaterials have been described (Pellegrino et al., 2005, Small, 1:48; Murray et al., 2000, Ann. Rev. Mat. Sci., 30:545; and Trindade et al., 2001, Chem. Mat., 13:3843). Additional methods have been described in the literature (see, e.g., Doubrow, Ed., “Microcapsules and Nanoparticles in Medicine and Pharmacy,” CRC Press, Boca Raton, 1992; Mathiowitz et al., 1987, J. Control. Release, 5:13; Mathiowitz et al., 1987, Reactive Polymers, 6:275; and Mathiowitz et al., 1988, J. Appl. Polymer Sci., 35:755; U.S. Pat. Nos. 5,578,325 and 6,007,845; P. Paolicelli et al., “Surface-modified PLGA-based Nanoparticles that can Efficiently Associate and Deliver Virus-like Particles” Nanomedicine. 5(6):843-853 (2010)).

Materials may be encapsulated into synthetic nanocarriers as desirable using a variety of methods including but not limited to C. Astete et al., “Synthesis and characterization of PLGA nanoparticles” J. Biomater. Sci. Polymer Edn, Vol. 17, No. 3, pp. 247-289 (2006); K. Avgoustakis “Pegylated Poly(Lactide) and Poly(Lactide-Co-Glycolide) Nanoparticles: Preparation, Properties and Possible Applications in Drug Delivery” Current Drug Delivery 1:321-333 (2004); C. Reis et al., “Nanoencapsulation I. Methods for preparation of drug-loaded polymeric nanoparticles” Nanomedicine 2:8-21 (2006); P. Paolicelli et al., “Surface-modified PLGA-based Nanoparticles that can Efficiently Associate and Deliver Virus-like Particles” Nanomedicine. 5(6):843-853 (2010). Other methods suitable for encapsulating materials into synthetic nanocarriers may be used, including without limitation methods disclosed in U.S. Pat. No. 6,632,671 to Unger issued Oct. 14, 2003.

In certain embodiments, synthetic nanocarriers are prepared by a nanoprecipitation process or spray drying. Conditions used in preparing synthetic nanocarriers may be altered to yield particles of a desired size or property (e.g., hydrophobicity, hydrophilicity, external morphology, “stickiness,” shape, etc.). The method of preparing the synthetic nanocarriers and the conditions (e.g., solvent, temperature, concentration, air flow rate, etc.) used may depend on the materials to be attached to the synthetic nanocarriers and/or the composition of the polymer matrix.

If synthetic nanocarriers prepared by any of the above methods have a size range outside of the desired range, synthetic nanocarriers can be sized, for example, using a sieve.

Elements of the synthetic nanocarriers may be attached to the overall synthetic nanocarrier, e.g., by one or more covalent bonds, or may be attached by means of one or more linkers. Additional methods of functionalizing synthetic nanocarriers may be adapted from Published US Patent Application 2006/0002852 to Saltzman et al., Published US Patent Application 2009/0028910 to DeSimone et al., or Published International Patent Application WO/2008/127532 A1 to Murthy et al.

Alternatively or additionally, synthetic nanocarriers can be attached to components directly or indirectly via non-covalent interactions. In non-covalent embodiments, the non-covalent attaching is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. Such attachments may be arranged to be on an external surface or an internal surface of a synthetic nanocarrier. In embodiments, encapsulation and/or absorption are a form of attaching.

Compositions provided herein may comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol).

Compositions according to the invention may comprise pharmaceutically acceptable excipients. The compositions may be made using conventional pharmaceutical manufacturing and compounding techniques to arrive at useful dosage forms. Techniques suitable for use in practicing the present invention may be found in Handbook of Industrial Mixing: Science and Practice, Edited by Edward L. Paul, Victor A. Atiemo-Obeng, and Suzanne M. Kresta, 2004 John Wiley & Sons, Inc.; and Pharmaceutics: The Science of Dosage Form Design, 2nd Ed. Edited by M. E. Auten, 2001, Churchill Livingstone. In an embodiment, compositions are suspended in sterile saline solution for injection with a preservative.

It is to be understood that the compositions of the invention can be made in any suitable manner, and the invention is in no way limited to compositions that can be produced using the methods described herein. Selection of an appropriate method of manufacture may require attention to the properties of the particular moieties being associated.

In some embodiments, compositions are manufactured under sterile conditions or are terminally sterilized. This can ensure that resulting compositions are sterile and non-infectious, thus improving safety when compared to non-sterile compositions. This provides a valuable safety measure, especially when subjects receiving the compositions have immune defects, are suffering from infection, and/or are susceptible to infection.

Administration according to the present invention may be by a variety of routes, including but not limited to subcutaneous, intravenous, and intraperitoneal routes. The compositions referred to herein may be manufactured and prepared for administration, in some embodiments concomitant administration, using conventional methods.

The compositions of the invention can be administered in effective amounts, such as the effective amounts described elsewhere herein. In some embodiments, the synthetic nanocarriers comprising an immunosuppressant and/or viral vectors are present in dosage forms in an amount effective to reduce an immune response and/or allow for readministration of a viral vector to a subject. In some embodiments, the synthetic nanocarriers comprising an immunosuppressant and/or viral vectors are present in dosage forms in an amount effective to escalate or achieve efficacious transgene expression in a subject. Dosage forms may be administered at a variety of frequencies. In some embodiments, repeated administration of synthetic nanocarriers comprising an immunosuppressant with a viral vector is undertaken.

Aspects of the invention relate to determining a protocol for the methods of administration as provided herein. A protocol can be determined by varying at least the frequency, dosage amount of the viral vector and synthetic nanocarriers comprising an immunosuppressant and subsequently assessing a desired or undesired immune response. A preferred protocol for practice of the invention reduces an immune response against the viral vector and/or the expressed product and/or promotes transgene expression. The protocol comprises at least the frequency of the administration and doses of the viral vector and synthetic nanocarriers comprising an immunosuppressant.

Another aspect of the disclosure relates to kits. In some embodiments, the kit comprises any one or more of the compositions provided herein. Preferably, the composition(s) is/are in an amount to provide any one or more doses as provided herein. The composition(s) can be in one container or in more than one container in the kit. In some embodiments of any one of the kits provided, the container is a vial or an ampoule. In some embodiments of any one of the kits provided, the composition(s) are in lyophilized form each in a separate container or in the same container, such that they may be reconstituted at a subsequent time. In some embodiments of any one of the kits provided, the kit further comprises instructions for reconstitution, mixing, administration, etc. In some embodiments of any one of the kits provided, the instructions include a description of any one of the methods described herein. Instructions can be in any suitable form, e.g., as a printed insert or a label. In some embodiments of any one of the kits provided herein, the kit further comprises one or more syringes or other device(s) that can deliver the composition(s) in vivo to a subject.

### EXAMPLES

### Example 1: Huh7 Studies

Differences between AAV2 and Anc80 reporter were examined using transfection studies. Huh7 cells were transduced infected with different constructs comprising engineered GFP (eGFP), and the resulting GFP was measured using FACS, 48 hours post AAV infection. The results are presented in Table 1.

TABLE 1
Results of FACS Analysis (Huh-7 Studies)

| | | GFP |
|---|---|---|
| | GFP + | Geometric |
| | (%) | Mean |
| Huh7 cell control, no infection | 0.74 | 6 |
| rAAV2-CB7-CI-eGFP-WPRE-rBG MOI 1E4 | 88.7 | 221 |
| AAV2/2-CMV-eGFP-WPRE.bGH (A646) MOI 1E4 | 90.3 | 297 |
| AAV2/2-CMV-eGFP-WPRE.bGH (A646) MOI 1E5 | 90.5 | 264 |
| AAV2/Anc80 AAP.-CMV-eGFP-WPRE.bGH | 90.2 | 177 |
| (A915) MOI 1E5 | | |
| AAV2/Anc80 AAP.-CMV-eGFP-WPRE.bGH | 90.4 | 385 |
| (A915) MOI 1E6 | | |
| AAV2/Anc80 AAP.-CMV-eGFP-WPRE.bGH | 92.5 | 491 |
| (A915) MOI 2E6 | | |

Note:
CB7, chicken β actin;
eGFP, engineered green fluorescent protein;
WPRE, Woodchuck hepatitis virus post-transcriptional regulatory element;
MOI, multiplicity of infection;
CMV, cytomegalovirus

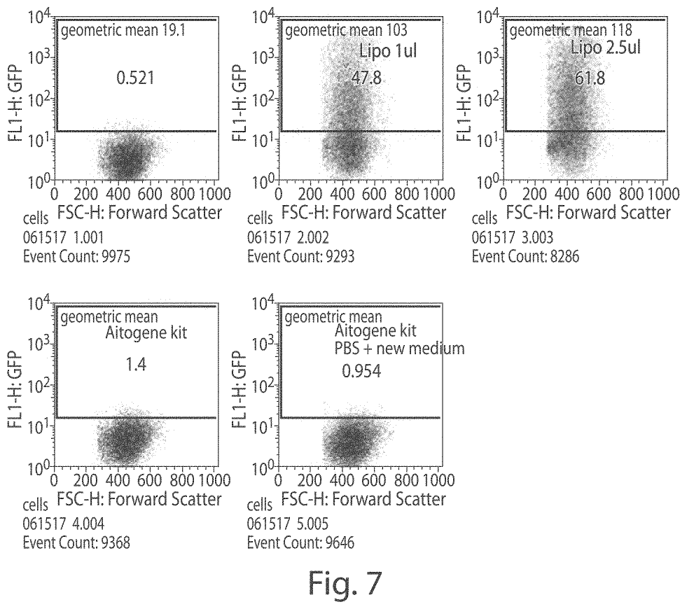

FIG. 7 shows an experiment comparing Lipo2000 and the Autogene Huh7 kit. The cells were grown in 24 well plates (1E4 per well), and transfected with 500 ng DNA EF1s-eGFP-WPRE/well. Forty-eight hours after transfection, the cells were assayed using FACS. The lipofectamine transfection resulted in 47-61% GFP cells.

### Example 2: synMUT4 Expression Studies

A series of Anc80 vector constructs expressing synthetic methyl malonyl CoA mutase 4 (synMUT4) were developed, including Anc80.CB7.synMUT4.RBG, Anc80.hAAT.synMUT4.RBG, Anc80.EF1s.synMUT4.HPRE, and Anc80.EF1s.synMUT4. C57BL6 (wild-type) mice, 8-10 weeks of age, underwent retro-orbital injections (systemic) of the constructs (5E+10), and were euthanized after 21 days. The experiments were conducted with five mice per group, with the exception of the Anc80.CB7.synMUT4.RBG group, which had four mice, due to a death during anesthesia. The control group did not receive injections. All major organs were collected.

Expression (mRNA) was determined using qPCR using specific primers and probe for synMUT4. GAPDH was used as an internal control, and levels were measured using ddPCR (BioRad).

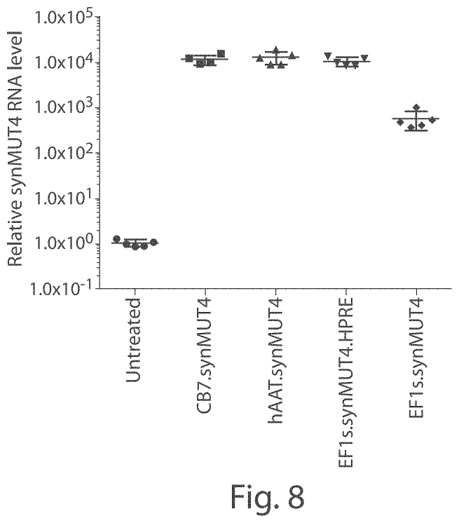

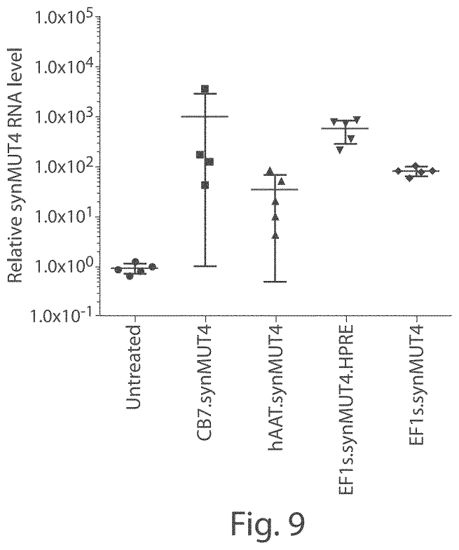

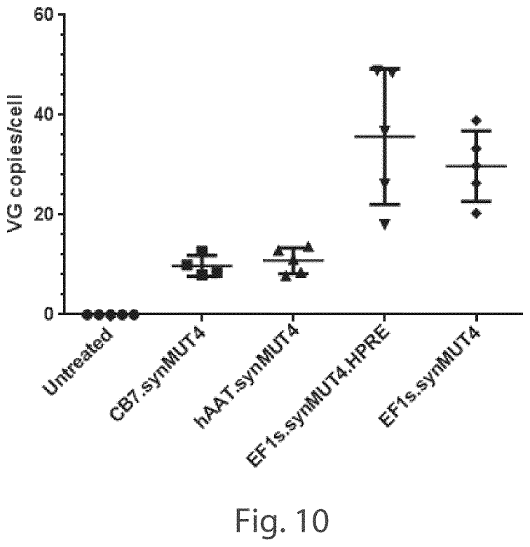

Expression was examined in liver (FIG. 8) and kidney (FIG. 9). The relative level of synMUT4 was increased in all of the constructs, compared to the untreated group. Further, biodistribution was examined in the liver (FIG. 10). The vector genomes per cell were found to be higher in the treatment groups, as compared to the untreated control group.

### Example 3: Analytical Ultracentrifugation Analysis

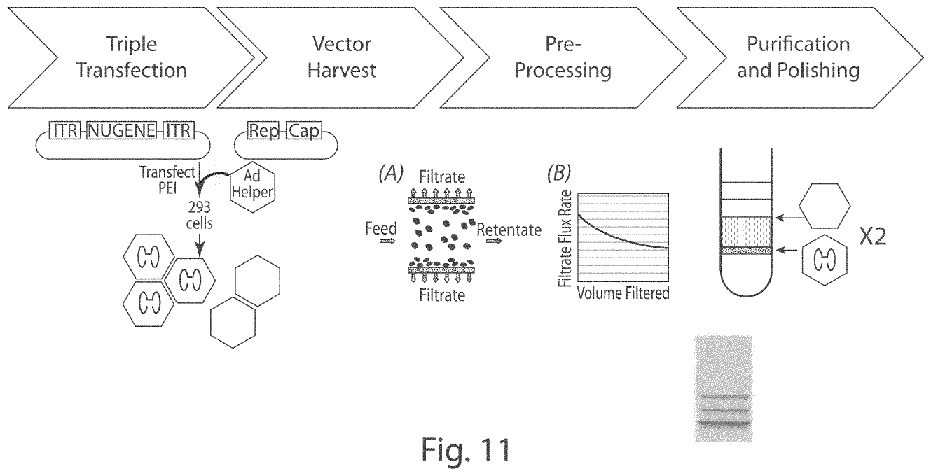

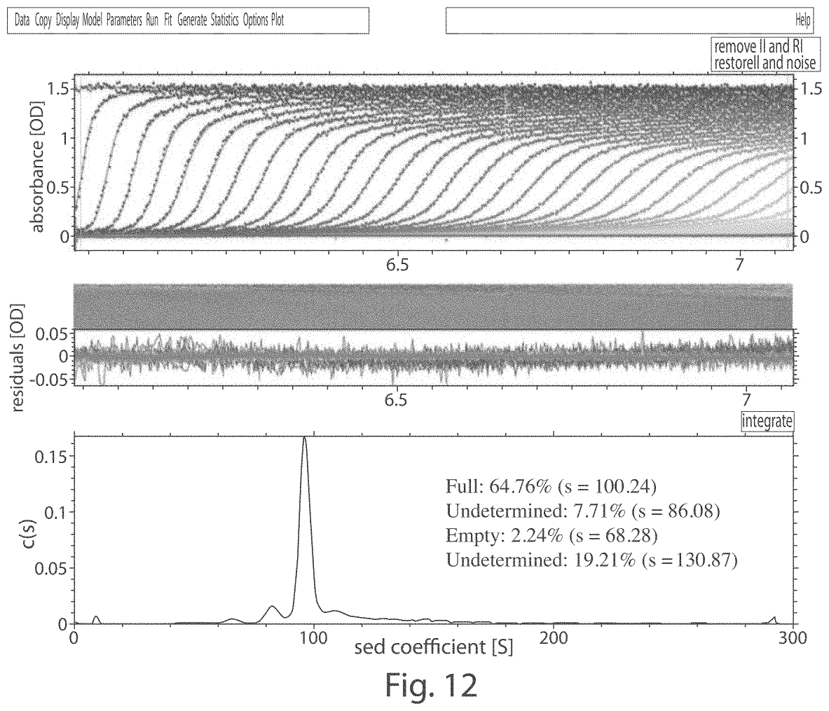

Analytical ultracentrifugation (AUC) was used to determine the reference standard for Anc80. As shown in FIG. 11, Hek293 cells were triple transfected with a vector (AAV2/Anc80 AAP.hAAT.synMUT4.RBG), harvested, and then pre-processed using filtration. The resulting filtrate was then centrifuged twice, which resulted in the formation of two layers, one comprising empty capsids, and the other comprising the vectors. The results were analyzed with Sedfit, and the data are presented in FIG. 12.

### Example 4: ImmTOR Particles are Well Tolerated in Mouse Models of MMA

The Mut^{-/-};Tg^{INS-MCK-Mut} mouse model (MUT) was used to study the effect of synthetic nanocarriers comprising rapamycin (ImmTOR nanoparticles) (Kishimoto, et al., 2016, Nat Nanotechnol, 11(10): 890-899; Maldonado, et al., 2015, PNAS, 112(2): E156-165). The MUT mice are deficient in methylmalonyl-CoA mutase in the liver, which is rescued from neonatal lethality by expression of the Mut gene in skeletal muscle under the control of a muscle creatine kinase (MCK) promoter. The MUT mice are a murine model of the severe juvenile form of MMA and manifest key clinical and biochemical features of methylmalonic acidemia (MMA), including growth retardation, susceptibility to dietary and environmental stress, highly elevated serum methylmalonic acid, and elevated fibroblast growth factor 21 (FGF21) (Manoli, et al., 2018, JCI Insight, 3(23): e124351). MUT mice respond to hepatic AAV gene therapy.

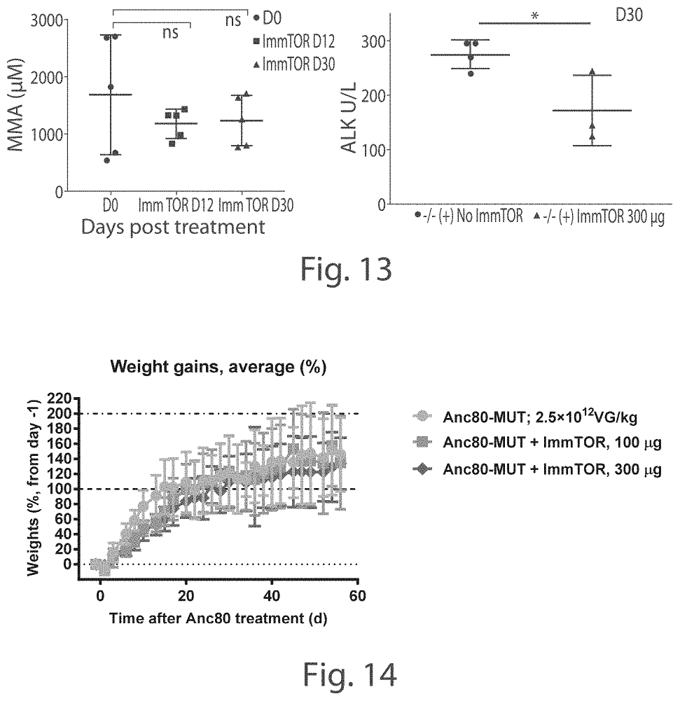

MUT mice have decreased liver function, including elevated alkaline phosphatase levels, relative to wild-type mice. MUT mice were administered the 300 μg ImmTOR nanoparticles and MMA and alkaline phosphatase levels were measured to determine if ImmTOR nanoparticles have any negative impact on liver function. MMA and alkaline phosphatase levels were stable, indicating that MUT mice tolerate high doses of ImmTOR (FIG. 13).

MUT mice were administered the Anc80-MUT vector (2.5×10¹² vg/kg) or co-administered the Anc80-MUT vector and 100 or 300 μg ImmTOR nanoparticles to determine if treatment with ImmTOR has any negative effect on weight in mice being treated with the Anc80-MUT vector. There was no significant difference in weight gain in MUT mice treated with the Anc80-MUT vector, the Anc80-MUT vector and 100 μg ImmTOR nanoparticles or the Anc80-MUT vector and 300 μg ImmTOR nanoparticles (FIG. 14). Thus, ImmTOR nanoparticles are well-tolerated in MUT mice.

### Example 5: ImmTOR Particles Decrease the Immune Response to Anc80 Vectors in Mouse Models of MMA

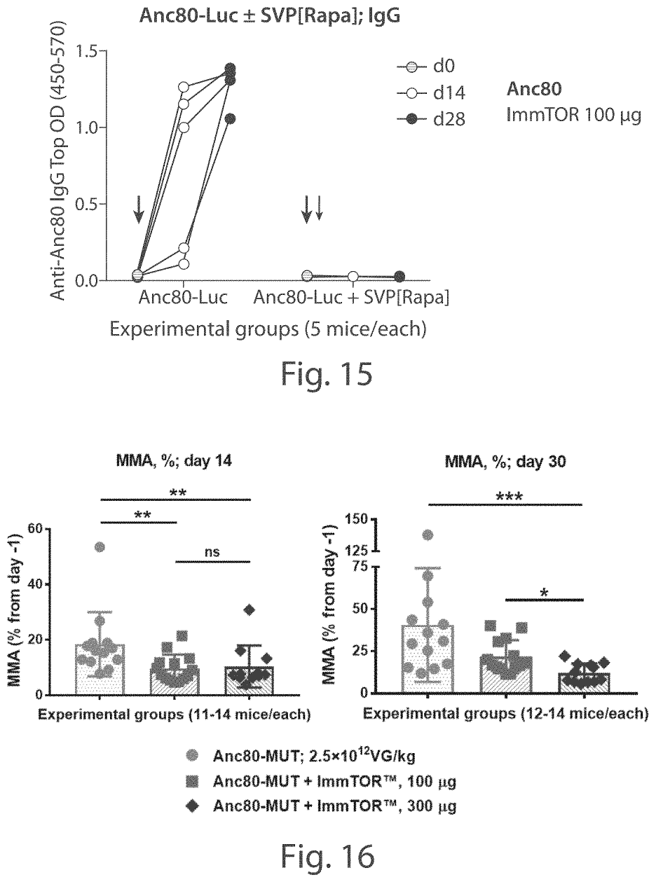

The effect of ImmTOR nanoparticles on the immune response to Anc80 vectors in MUT mice was examined. MUT deficient mice administered the Anc80 vector develop antibodies to Anc80. These antibodies can neutralize the therapeutic effects of Anc80 vectors. MUT mice were administered the Anc80-CB-luciferase vector (5.0×10¹⁰ vg/kg) or co-administered the Anc80-luciferase vector (5.0×10¹⁰ vg/kg) and 300 μg ImmTOR nanoparticles. Anti-Anc80 antibody levels were measured 14 and 28 days after administration. A decrease in anti-Anc80 antibodies was observed at both time points in mice co-administered the Anc80-luciferase vector (5.0×10¹⁰ vg/kg) and 300 μg ImmTOR nanoparticles relative to mice administered the Anc80-luciferase vector alone (FIG. 15). The results presented herein demonstrate that concomitant administration of ImmTOR nanoparticles inhibits the formation of anti-Anc80 antibodies in mice treated with Anc80 vectors.

### Example 6: ImmTOR Particles Increase Efficacy of Anc80-MUT Vectors in Mouse Models of MMA

MUT mice were administered the Anc80-MUT vector (2.5×10¹² vg/kg) or co-administered the Anc80-MUT vector and 100 or 300 μg ImmTOR nanoparticles to determine if co-administration of ImmTOR nanoparticles and the Anc80-MUT vector reduces serum methylmalonic acid (MMA). Fourteen days after administration of the Anc80-MUT vector or the Anc80-MUT vector and ImmTOR nanoparticles, there was a significant decrease in MMA levels in MUT mice co-administered the Anc80-MUT vector and ImmTOR nanoparticles (both 100 μg and 300 μg) compared to MUT mice administered the Anc80-MUT vector alone (FIG. 16). Thirty days after administration of the Anc80-MUT vector or the Anc80-MUT vector and ImmTOR nanoparticles, there was a significant decrease in MMA levels in MUT mice co-administered the Anc80-MUT vector and 300 μg ImmTOR nanoparticles compared to MUT mice administered the Anc80-MUT vector alone (FIG. 16). The results presented herein demonstrate that concomitant administration of ImmTOR nanoparticles decreases expression of MMA in mice treated with Anc80-MUT vectors.

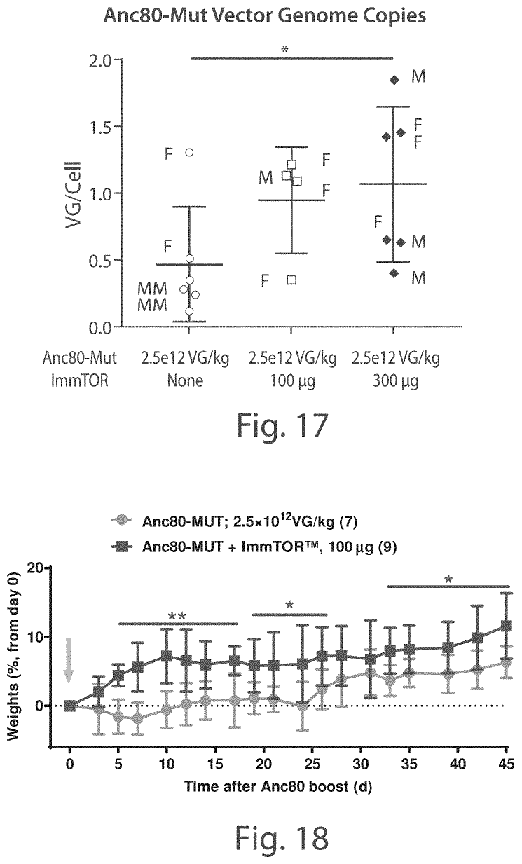

Additionally, MUT mice were administered the Anc80-MUT vector (2.5×10¹² vg/kg) or co-administered the Anc80-MUT vector and 100 or 300 μg ImmTOR nanoparticles to determine if co-administration of ImmTOR nanoparticles increases Anc80-MUT vector genomes in the liver. Anc80-MUT DNA levels were measured by quantitative PCR using MUT-specific primers 30 days after administration or co-administration. There was a dose-dependent increase of vector genome copy number with co-administration of the Anc80-MUT vector and ImmTOR nanoparticles (FIG. 17) relative to administration of the Anc80-MUT vector alone. The results of this experiment demonstrate that concomitant administration of ImmTOR nanoparticles increases Anc80-MUT genome number in the liver in mice treated with Anc80-MUT vectors.

Taken together, the results from this example show that a single concomitant administration of ImmTOR and the Anc80-MUT vector in a mouse model of MMA increases efficacy of the Anc80-MUT vector.

### Example 7: Repeat Administration of ImmTOR Particles Increases Efficacy of Anc80-MUT Vectors in Mouse Models of MMA

MUT mice administered a first dose of the Anc80-MUT vector or the Anc80-MUT vector and ImmTOR nanoparticles were administered a second dose of the Anc80-MUT vector or co-administered the Anc80-MUT vector and ImmTOR nanoparticles to examine the tolerability and efficacy of a second dose.

First, MUT mice administered a first dose of the Anc80-MUT vector or the Anc80 vector and ImmTOR nanoparticles on day 0 were administered a second dose of the Anc80-MUT vector or co-administered the Anc80-MUT vector and 100 μg ImmTOR nanoparticles on day 56 and the weight of the mice was followed after the second administration. MUT mice co-administered a second dose of the ImmTOR nanoparticles and the Anc80-MUT vector had a significant, early weight gain benefit compared with MIT mice administered the Anc80-MUT vectors only (FIG. 18).

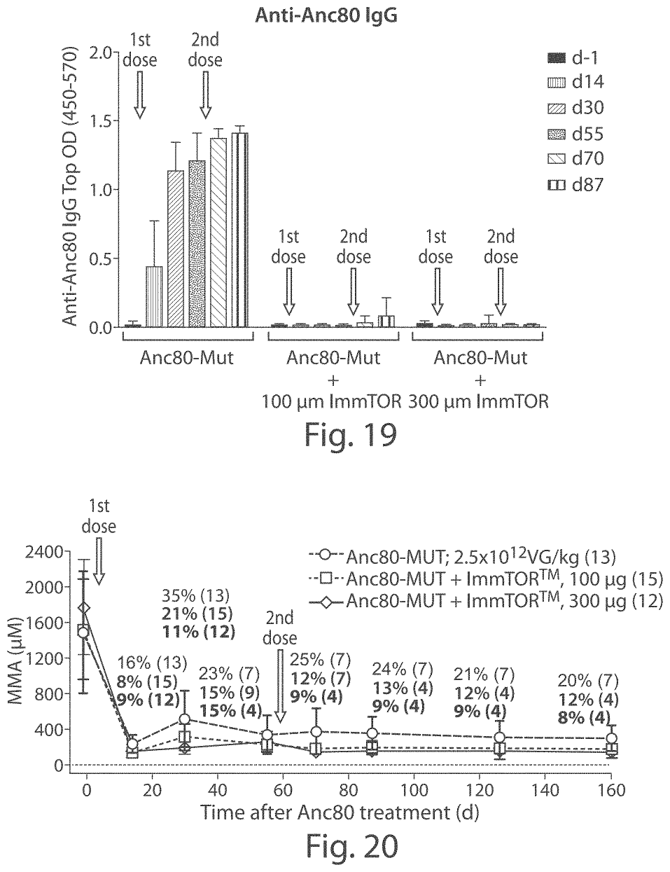

MUT mice administered a first dose of the Anc80-MUT vector or the Anc80 vector and ImmTOR nanoparticles on day 0 were administered a second dose of the Anc80-MUT vector or co-administered the Anc80-MUT vector and 100 or 300 μg ImmTOR nanoparticles on day 57 and anti-Anc80 antibody levels were measured to determine if repeat co-administration of ImmTOR with Anc80-MUT inhibits the formation of anti-Anc80 antibodies. MUT mice administered the Anc80-MUT vector develop antibodies to Anc80 after a single administration at day 0 and following the second administration at day 56. In MUT co-administered the Anc80-MUT vector and 100 or 300 μg ImmTOR nanoparticles, the levels of anti-Anc80 antibodies did not increase following the first dose. For the 300 μg group, levels of anti-Anc80 antibodies also did not increase after the second dose. For the 100 μg group, anti-Anc80 antibody levels remained reduced relative to Anc80-MUT treated mice up to 87 days. (FIG. 19). The results presented herein demonstrate that concomitant administration of ImmTOR nanoparticles inhibits the formation of anti-Anc80 antibodies in mice treated with Anc80-MUT vectors after a first and second dose.

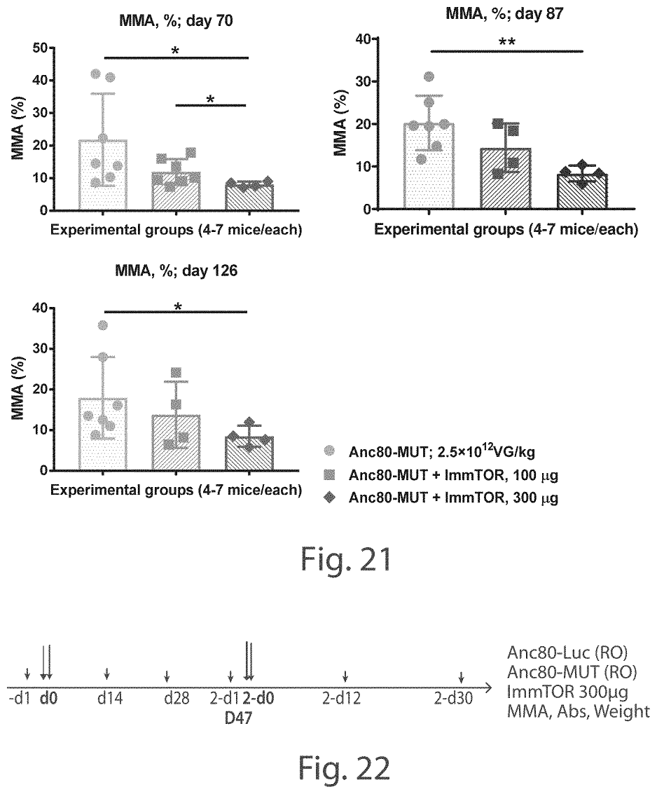

Serum MMA levels were also measured after MUT mice were administered a first dose of the Anc80-MUT vector or the Anc80 vector and ImmTOR nanoparticles on day 0 and were administered a second dose of the Anc80-MUT vector (2.5×10¹² vg/kg) or co-administered the Anc80-MUT vector and 100 or 300 μg ImmTOR nanoparticles on day 56. Administration of a second dose of ImmTOR nanoparticles with the Anc80-MUT vector on day 56 after the first dose leads to a dose-dependent, additional decrease in serum MMA levels compared to administration of Anc80-MUT vector only (FIGS. 20, 21). The results presented herein demonstrate that concomitant administration of ImmTOR nanoparticles inhibits MMA expression in mice treated with the Anc80-MUT vectors after a first and second dose.

### Example 8: Repeat Administration of ImmTOR Particles Increases Efficacy of Anc80-MUT Vectors in Mouse Models of MMA

MUT mice administered a dose of the Anc80-CB-Luc vector or the Anc80-CB-Luc vector and ImmTOR nanoparticles were later administered a dose of the Anc80-MUT vector or co-administered the Anc80-MUT vector and ImmTOR nanoparticles to examine the tolerability and efficacy of a second dose with a different Anc80 vector.

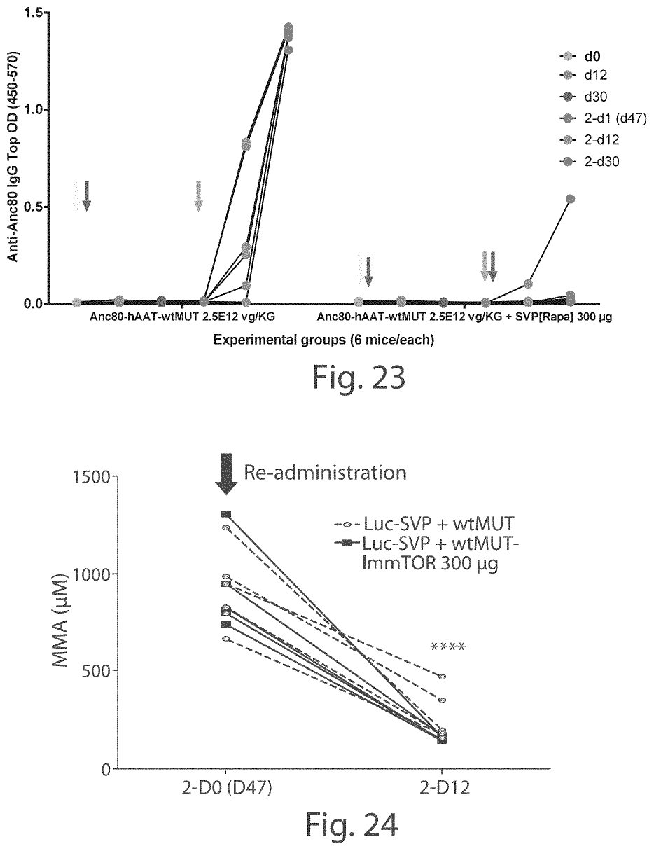

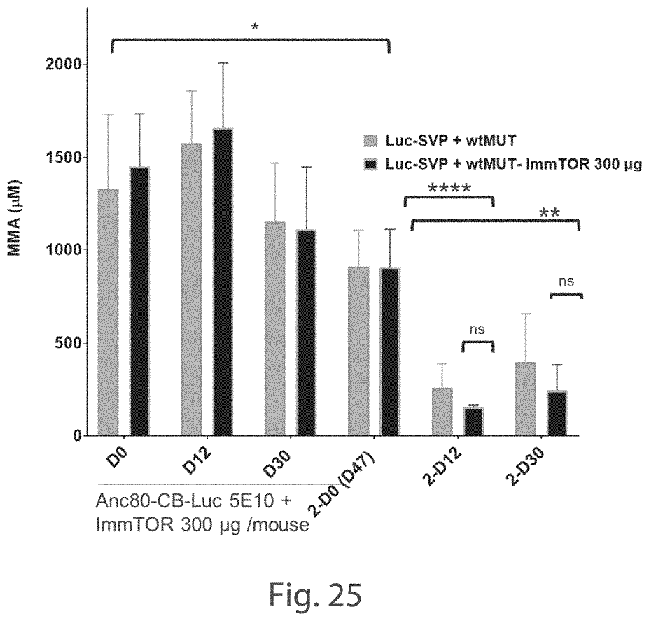

MUT mice were administered a first dose of the Anc80-CB-Luc 5E10 and 300 μg ImmTOR nanoparticles on day 0 and then were administered the Anc80-MUT vector or co-administered the Anc80-MUT vector and 300 μg ImmTOR nanoparticles on day 47 (FIG. 22). Anti-Anc80 antibody levels were measured to determine if co-administration of ImmTOR with Anc80-MUT inhibits the formation of anti-Anc80 antibodies after a first administration of Anc80-CB-Luc with ImmTOR nanoparticles. The levels of anti-Anc80 antibodies were decreased in MUT mice co-administered the Anc80-MUT vector and ImmTOR nanoparticles up to at least 30 days after administration of the second dose (FIG. 23). Thus, administration of ImmTOR nanoparticles with Anc80-MUT vector inhibits formation of anti-Anc80 antibodies after a first administration of ImmTOR nanoparticles and a different Anc80 vector. MMA levels were also measured in MUT mice treated according to the same protocol. The levels of MMA were significantly decreased in MUT mice co-administered the Anc80-MUT vector and ImmTOR nanoparticles after administration of Anc80-CB-Luc 5E10 and ImmTOR nanoparticles (FIGS. 24, 25). Thus, administration of ImmTOR nanoparticles with Anc80-MUT after a first administration of ImmTOR nanoparticles and a different Anc80 vector decrease the serum levels of MMA.

### Example 9: Repeated High Doses of ImmTOR Particles Provides Therapeutic Efficacy

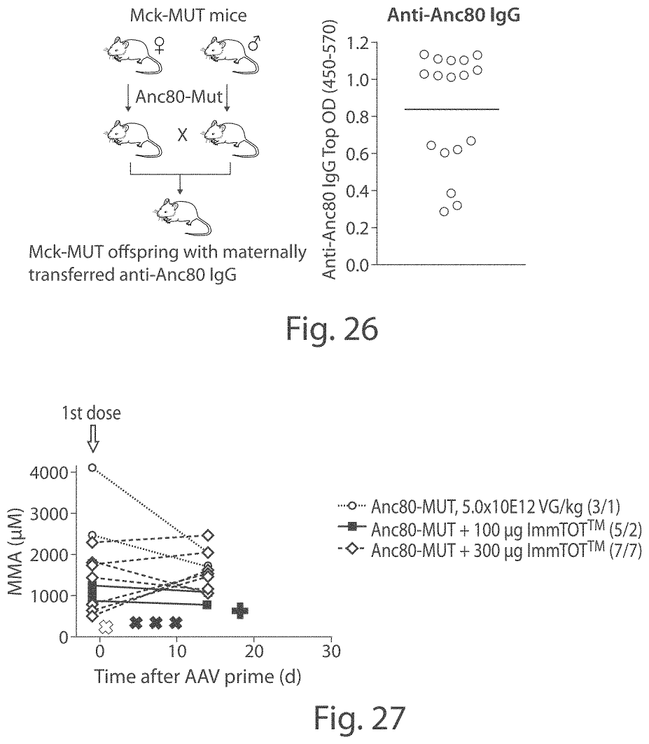

Male and female MUT mice were administered the Anc80-MUT vector to generate anti-Anc80 antibodies. The mice with anti-Anc80 antibodies were crossed to generate MUT mice with maternally-transferred anti-Anc80 antibodies (FIG. 26).

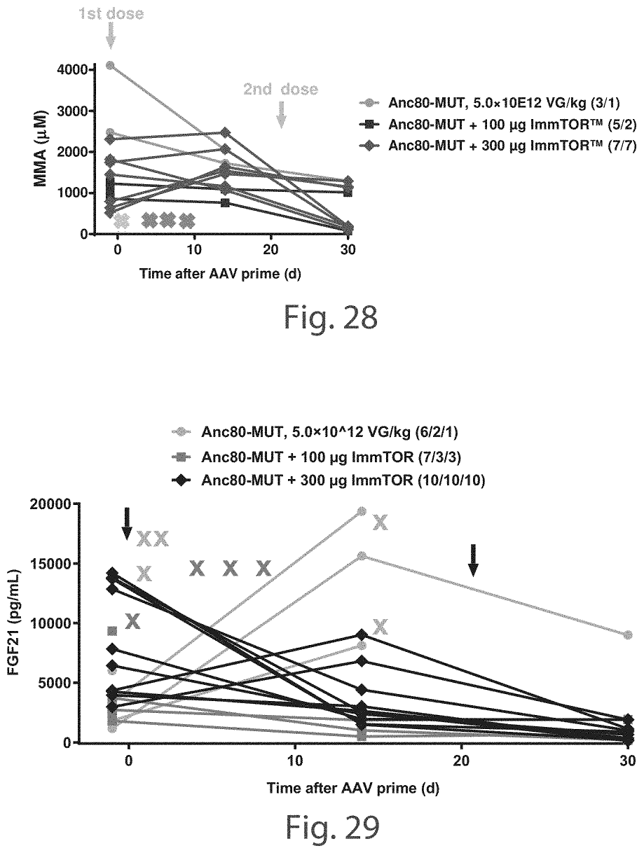

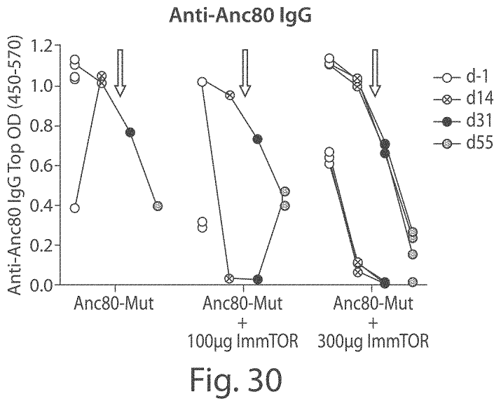

MUT mice with maternally-transferred anti-Anc80 antibodies were administered a high dose of the Anc80-MUT vector (5.0×10¹² vg/kg) or co-administered the Anc80-MUT vector (5.0×10¹² vg/kg) and 100 or 300 μg ImmTOR nanoparticles to examine if the co-administration of the ImmTOR nanoparticles with the Anc80-MUT vector mitigated the effect of the anti-Anc80 antibodies on MMA levels (FIG. 27). Although there was no clinically meaningful reduction on serum MMA levels in MUT mice, 7 of 7 MUT mice administered the 300 μg ImmTOR nanoparticles survived to 14 days after administration, whereas only 2 of 5 MUT mice administered the 100 μg ImmTOR nanoparticles and 2 of 3 MUT mice administered only the Anc80 AAV vector survived to 14 days after administration (FIG. 21). When the mice were administered a second dose of the Anc80-MUT vector (5.0×10¹² vg/kg) or the Anc80-MUT vector (5.0×10¹² vg/kg) and 100 or 300 μg ImmTOR nanoparticles, the 5/7 surviving mice MUT mice administered a second dose of the Anc80-MUT vector and 300 μg ImmTOR nanoparticles showed reduced serum MMA levels (FIGS. 28, 30). Similar effects were seen with respect to the levels of fibroblast growth factor 21 (FGF21), an inflammatory cytokine shown to correlate with MMA severity (Manoli et al., JCI Insight. 2018; 3(23):e124351) with both 1st and 2nd dose of Anc80-MUT combined with 300 μg of ImmTOR resulting in reduced FGF21 levels in MUT mice. FIG. 29).

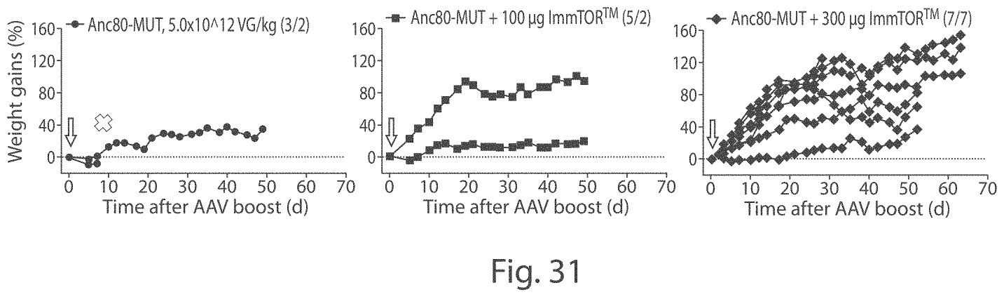

Additionally, weight gain was examined in the MUT mice with maternally-transferred anti-Anc80 antibodies that were administered a second dose of the Anc80-MUT vector (5.0×10¹² vg/kg) or co-administered the Anc80-MUT vector and 100 or 300 μg ImmTOR nanoparticles. Two of three MUT mice administered the Anc80-MUT vector survived to receive the second administration, but one of these mice died shortly thereafter (FIG. 31). Only two of five MUT mice administered the Anc80-MUT vector and 100 μg ImmTOR nanoparticles survived to receive the second administration, and only one of the two surviving mice increased weight after the second administration (FIG. 31). In contrast, seven of seven MUT mice administered the Anc80-MUT vector and 300 μg ImmTOR nanoparticles survived to receive the second administration, and six of the seven mice increased weight after the second administration (FIG. 31).

Thus, pre-existing humoral immunity in MUT mice with maternally-transferred anti-Anc80 antibodies leads to sub-optimal therapeutic performance of the Anc80-MUT vector after a first administration. High doses (300 μg) of ImmTOR nanoparticles administered concomitantly with high doses of the Anc80-MUT vector increase survival after a first administration and provide therapeutic efficacy after repeated administration.

### Example 10: Incidence of Antibodies Against Anc80 in MMA Patients

Levels of anti-Anc80 antibodies were measured in MMA patients aged 2-32 years. The results are presented in Table 2 below. The results presented herein show that MMA patients have a low incidence of antibodies against Anc80.

TABLE 2
Incidence of antibodies against Anc80 in MMA patients

| MMA MUT | AGE | TRANSPLANT | Anc-80 | Anc-80 |
|---|---|---|---|---|
| PATIENTS | RANGE | STATUS | SEROPOSITIVE* | SERONEGATIVE* |
| TOTAL | 2-32 YR | UNTRANSPLANTED | 2/27 | 25/27 |
| N = 33 | | N = 27 | | |
| | | TRANSPLANTED | 3/6 | 3/6 |
| | | N = 6 | | |

1. A method comprising:
concomitantly administering a viral vector such as an AAV vector or Anc80 vector to a subject that has or is suspected of having an organic acidemia and synthetic nanocarriers coupled to an immunosuppressant, wherein the viral vector comprises a sequence that encodes an enzyme associated with the organic acidemia and one or more expression control sequences.

2. The method of claim 1, wherein the viral vector is an AAV2, AAV8, Anc80, AAV2/Anc80 or AAV8/Anc80 vector.

3. The method of claim 1, wherein the organic acidemia is methylmalonic acidemia (MMA).

4. The method of claim 1, wherein the viral vector and synthetic nanocarriers coupled to an immunosuppressant are in an amount effective to reduce humoral and/or cellular immune responses to the viral vector.

5. (canceled)

6. The method of claim 1, wherein the subject has been previously administered the viral vector and synthetic nanocarriers coupled to an immunosuppressant concomitantly.

7. The method of claim 1, wherein the method further comprises administering the viral vector to the subject at a subsequent point in time.

8. The method of claim 1, wherein the concomitant administration of the viral vector and synthetic nanocarriers coupled to an immunosuppressant is repeated.

9. The method of claim 1, wherein the enzyme associated with the methylmalonic acidemia (MMA) is methylmalonyl-CoA mutase (MUT).

10. The method of claim 1, wherein the sequence encodes a wild-type MUT.

11. The method of claim 1, wherein the one or more expression control sequences comprises a liver-specific promoter.

12-38. (canceled)

39. The method of claim 1, wherein the subject is in need of liver or kidney expression of the enzyme.

40. The method of claim 39, wherein the subject is administered an AAV8 vector or Anc80 vector comprising a sequence encoding the enzyme and a liver-specific promoter or a constitutive promoter.

41. The method of claim 1, wherein the subject is in need of kidney expression of the enzyme, and the subject is administered an Anc80 vector encoding the enzyme and a constitutive promoter.

42-44. (canceled)

45. A composition comprising:
a dose of a viral vector of claim 1.

46. The composition of claim 45, wherein the composition further comprises a dose of synthetic nanocarriers of claim 1.

47. The composition of claim 45 wherein the composition is a kit.

48-49. (canceled)

50. A composition comprising a viral vector of claim 1.

51. A composition comprising a nucleic acid encoding the sequence of claim 1.

52. A composition comprising a viral vector comprising the nucleic acid of claim 51.

53. A composition comprising an AAV2, AAV8, Anc80, AAV2/Anc80 or AAV8/Anc80 vector comprising a sequence that encodes the enzyme of claim 1 with a liver-specific promoter or a constitutive promoter.

54-59. (canceled)
